(19) United States
(12) Patent Application Publication
(10) Pub. No.: US 2010/0330607 A1
(43) Pub. Date: Dec. 30, 2010

(54) PHOTOSWITCH-ENABLED ION CHANNEL ASSAY SYSTEM

(75) Inventors: Jay Trautman, Los Altos, CA (US); Andrew Blatz, San Meteo, CA (US)

Correspondence Address: WILSON, SONSINI, GOODRICH & ROSATI, 650 PAGE MILL ROAD, PALO ALTO, CA, 94304-1050, US

(73) Assignee: Photoswitch Biosciences, Inc., San Francisco, CA (US)

(21) Appl. No.: 12/822,101

(22) Filed: Jun. 23, 2010

Related U.S. Application Data
(60) Provisional application No. 61/220,165, filed on Jun. 24, 2009.

(51) Int. Cl. C12Q 1/02; C12M 1/34; H01J 40/14

(52) U.S. Cl. 435/29; 435/288.7; 250/578.1

(57) ABSTRACT

The present invention provides a system for assaying ion-channels. In some embodiments, the ion-channel assay provides a reversible change to the membrane potential of a target, e.g., a cell, upon exposure to light. In some embodiments, the membrane potential readout is fed back through a control circuit to regulate the excitation intensity of the illumination sources that induce, respectively, hyperpolarizing and depolarizing currents, thereby effecting closed-loop control of the membrane potential.

### CROSS-REFERENCE

This application claims the benefit of U.S. Provisional Application No. 61/220,165, filed Jun. 24, 2009, which application is incorporated herein by reference.

### STATEMENT AS TO FEDERALLY SPONSORED RESEARCH

This invention was made with government support under Small Business Innovation Research Awards for “Ion Channel Drug Discovery Using Photoswitch Technology” (award number 1R43GM087755-01A2) and “Pharmaceutical Therapy for Seasonal Affective Disorder” (award number 1R43MH088062-01A1) awarded by the National Institutes of Health. The government has certain rights in the invention.

### BACKGROUND OF THE INVENTION

Ion channels are a diverse set of proteins that are broadly classified by the mechanism of activation into voltage-gated and ligand-gated channels. Voltage-gated channels open and close, in a membrane-potential-dependent manner, meaning that these proteins change conformation in response to a change in the local electric field, resulting in a change in the channel conductance. Ligand-gated ion channels, also known as ionotropic receptors, are a group of channels that open in response to the binding of specific molecules to the extracellular domain of the receptor protein. Ligand binding causes a conformational change in the structure of the channel protein that ultimately leads to a change in the state of the channel gate and the consequent ion flux across the plasma membrane.

The canonical voltage-gated channel is described as having three states: open, closed and inactivated. Compounds can affect channels differently in the various states, and their effects can increase on repeated membrane depolarization. These two properties are termed state- and use-dependence, and assaying compounds for state- and/or use-dependence facilitates the discovery and development of ion channel therapeutics. Examples of state- and use-dependence are provided in the following sections:

Block by local anesthetics (LA) depends on the history, as well as the value, of the cellular membrane potential. In general, the block is relatively weak if the membrane is at its resting potential, and it is strong when the membrane is depolarized. In addition, the block is more effective if the membrane is repeatedly depolarized. This phasic block has been termed “use dependent.” The modulated receptor hypothesis assumes that affinity for LA depends on the conformational state of the channel with the inactivated state showing the highest affinity. This energetically favored conformation is stabilized in the presence of LA, resulting in a shift of the steady-state inactivation curve to more negative potentials. A different interpretation (“guarded receptor hypothesis”) assumes that drug binds to a constant-affinity receptor whose access is regulated by channel gates leading to apparently variable affinities. Irrespective of mechanism, the property of use dependence is an important attribute of successful LAs.

The human ether-a-go-go-related (hERG) channels give rise to an important K+ current during the cardiac cycle and have been implicated as a major determinant of drug development failure due to safety concerns deriving from the cardiac effects. The FDA currently mandates functional screening of drug candidates against this channel. hERG channels pass through at least three kinetic states during activity: open, closed, and inactivated. In response to depolarization from the resting membrane potential the channel slowly enters the open state, and then rapidly moves to an inactivated state. Thus, very little current is observed to this simple depolarization. However, upon stepping from this depolarized potential to a modestly hyperpolarized voltage, the channel rapidly moves from the inactivated state to the open state, revealing large easily-measured currents before finally entering the closed state. Two-step voltage protocols such as this have made use of a patch clamp technique. To evoke measurable currents from these channels, the membrane potential is first depolarized, and then a hyperpolarizing step is used to bring the membrane to a potential negative enough to begin to close channels, but still depolarized relative to the electrochemical equilibrium potential of K+. In response to this step, the channels rapidly enter the open conformational state and then slowly close.

Failure to maintain Na+ and Ca2+ homeostasis can lead to arrhythmia and reduced contractility, mitochondrial dysfunction and eventually, cell death. On depolarization, inward sodium current (INa) increases rapidly, reaching a transient peak lasting a few ms, before beginning to inactivate. The inactivation of INa has a fast component that lasts a couple of ms followed by a slow component that can last hundreds of ms. An enhanced late INa (that due to the slow component) may contribute to the Na overload observed in ischemia and heart failure (HF). The drug compound ranolazine causes a concentration-, voltage- and frequency-dependent inhibition of the late INa on ventricular myocytes from dog and guinea-pig hearts. Ranolazine is approved for use in the treatment of chronic angina pectoris. The efficacy of the drug may be due to its indirectly preventing Ca2+ overload that causes cardiac ischemia, by directly inhibiting the late Na+ current.

The dihydropyridine class of antihypertensives produces vasodilation through inhibition of Cav1.2 L-type channel in vascular smooth muscle without excessive negative inotropy from inhibition of its target in ventricular muscle. The tissues have different resting potentials: that of vascular smooth muscle is typically −40 mV, whereas that of heart is −85 mV. Consequently, the target channels in smooth muscle have higher fractional inactivation at rest than those in heart and, as it is the inactivated channel that is the target of this class of drug, the block is more thorough in smooth muscle.

Many forms of inflammatory and neuropathic pain result from hyperexcitable nociceptors. Use dependence can be responsible for differential efficacy on the target cells compared to off-target cells possessing the target channel. The compound is more effective with repeated activation of the target channel, and that channel is more active in the target cell type than in off-target cells. Alternately, ziconotide blocks Cav2.2 with little use dependence, and correspondingly it has a narrow Therapeutic Index.

There is a need for new technologies that provide rapid and cost-effective identification of compounds that affect the important physiological functions of ion channels including state- and use-dependent conductance.

### SUMMARY OF THE INVENTION

In one aspect, the present invention provides a method for determining the effect of a compound on a test channel, the method comprising: providing cells expressing both the test channel and a photoswitchable ion channel; contacting the cells with a photoswitch regulator of the photoswitchable ion channel; illuminating the contacted cells with a first light source that modulates the photoswitchable ion channel; and determining the effect of the compound on the test channel.

In some embodiments, determining the effect of the compound on the test channel comprises determining a membrane potential of the cells. In some embodiments, determining the effect of the compound on the test channel comprises determining a change in the concentration of an ion.

In some embodiments, the compound is an agonist, an antagonist, an allosteric modulator, or a blocker of the test channel. In some embodiments, the compound comprises a small-molecule, a polypeptide, or a nucleic acid.

In some embodiments, the test channel is exogenous to the cell. In some embodiments, the test channel is a voltage-dependent ion channel. In some embodiments, the voltage-dependent ion channel is a voltage-gated ion channel or a ligand-gated ion channel.

In some embodiments, the cells further express one or more proteins that extend the range of the membrane potential changes resulting from the activity of the photoswitchable channel. In some embodiments, the one or more proteins comprise an inward rectifier channel. In some embodiments, the inward rectifier channel comprises a Kir channel. In some embodiments, the one or more proteins comprise an active transport. In some embodiments, the active transport comprises a Na+/K+ ATPase.

In some embodiments, the photoswitchable ion channel is exogenous to the cell. In some embodiments, the photoswitch regulator comprises a photoisomerizable moiety and one or more protein association moieties. In some embodiments, the photoswitch regulator comprises a photochromic ligand. In some embodiments, the photoisomerizable moiety comprises an azobenzene, a fulgide, a spiropyran, a triphenyl methane, a thioindigo, a diarylethene, or an overcrowded alkene. In some embodiments, at least one protein association moiety forms a covalent bond with the photoswitchable ion channel. In some embodiments, at least one protein association moiety forms a non-covalent bond with the photoswitchable ion channel. In some embodiments, at least one protein association moiety comprises a ligand that binds to a ligand-binding site or an allosteric site of the photoswitchable ion channel.

In some embodiments, the cells are illuminated for between 1 to 10 ms. In some embodiments, modulation of the photoswitchable ion channel by the first light source comprises partial or complete stimulation, activation, inactivation, or block. In some embodiments, the cells are illuminated with a second light source after illuminating the cells with the first light source. In some embodiments, the cells are illuminated by the second light source for between 1 to 100 ms. In some embodiments, illumination by the second light source counteracts the modulation of the photoswitchable ion channel by the first light source. In some embodiments, illumination of the cells is repeatedly alternated between the first and second light sources. In some embodiments, illumination of the cells by the first and second light sources is cycled in a sequential manner, an overlapping manner, or a combination thereof.

In some embodiments, determining the membrane potential of the cells comprises one or more of an optical measurement and an electrical measurement. In some embodiments, the optical measurement comprises detecting a voltage-sensitive dye fluorescence, a voltage sensitive fluorescence resonance energy transfer (FRET), or a nanocrystal luminescence. In some embodiments, determining a change in the concentration of an ion comprises detecting an ion sensitive dye fluorescence.

In some embodiments, an error signal is produced when the determined membrane potential differs from an expected membrane potential range. In some embodiments, the expected membrane potential is set within the range of the photoswitchable ion channel, or about −100 mV to about +50 mV. In some embodiments, the error signal modulates one or more light sources. In some embodiments, modulating the one or more light sources comprises modulating illumination intensity to increase depolarizing currents and/or hyperpolarizing currents.

In some embodiments, the method is performed using a microtiter plate. For example, a 96-well plate or a 384-well plate can be used. In some embodiments, the method is performed using a dispenser configured to dispense one or more reagents to the microtiter plate.

In some embodiments, the photoswitchable ion channel comprises a Synthetic Photoisomerizable Azobenzene-Regulated K+ (SPARK) channel or a variant thereof and the photoswitch regulator comprises Maleimide-Azobenzene-Quatenary ammonium (MAQ) or a variant thereof. In some embodiments, the photoswitchable ion channel comprises a glutamate receptor. In some embodiments, the photoswitchable ion channel comprises a Light-activated ionotropic Glutamate Receptor (LiGluR) channel or a variant thereof and the photoswitch regulator comprises Maleimide-Azobenzene-Glutamate (MAG) or a variant thereof. In some embodiments, the photoswitchable ion channel comprises a sodium channel. In some embodiments, the photoswitchable ion channel comprises an epithelial sodium channel (ENaC). In some embodiments, the photoswitch regulator of the sodium channel comprises a Maleimide-Azobenzene-Amiloride (MAA) or a variant thereof. In some embodiments, the ENaC is blocked by triamterene or amiloride.

In one aspect, the present invention provides a system for determining the effect of a compound on a test channel, the system comprising: cells expressing the test channel and a photoswitchable ion channel; a photoswitch regulator of the photoswitchable ion channel; one or more illumination sources; and a device configurable to determine the effect of the compound on the test channel.

In some embodiments, determining the effect of the compound on the test channel comprises determining a membrane potential of the cells. In some embodiments, determining the effect of the compound on the test channel comprises determining a change in the concentration of an ion.

In some embodiments, the test channel is exogenous to the cells. In some embodiments, the test channel is a voltage-dependent ion channel. In some embodiments, the voltage-dependent ion channel is a voltage-gated ion channel or a ligand-gated ion channel.

In some embodiments, the cells further express one or more proteins that extend the range of membrane potential changes. In some embodiments, the one or more proteins comprise an inward rectifier channel. In some embodiments, the inward rectifier channel comprises a Kir channel. In some embodiments, the one or more proteins comprise an active transport. In some embodiments, the active transport comprises a Na+/K+ ATPase.

In some embodiments, the compound comprises a small-molecule, a polypeptide, or a nucleic acid.

In some embodiments, the photoswitchable ion channel is exogenous to the cell. In some embodiments, the photoswitch regulator comprises a photoisomerizable moiety and one or more protein association moieties. In some embodiments, the photoisomerizable moiety comprises an azobenzene, a fulgide, a spiropyran, a triphenyl methane, a thioindigo, a diarylethene, or an overcrowded alkene. In some embodiments, at least one protein association moiety forms a covalent bond with the photoswitchable ion channel. In some embodiments, at least one protein association moiety forms a non-covalent bond with the photoswitchable ion channel. In some embodiments, at least one protein association moiety comprises a ligand that binds to a ligand-binding site or an allosteric site of the photoswitchable ion channel.

In some embodiments, the device is configurable to determine one or more of an optical measurement and an electrical measurement. In some embodiments, the optical measurement comprises detecting a voltage-sensitive dye fluorescence, a voltage sensitive fluorescence resonance energy transfer (FRET), a nanocrystal luminescence, or an ion-sensitive dye fluorescence.

In some embodiments, the device is configurable to produce an error signal when the determined membrane potential differs from an expected membrane potential range. In some embodiments, the expected membrane potential is set within the range of the photoswitchable ion channel, or about −100 mV to about +50 mV. In some embodiments, the system is configurable to modulate one or more light sources when the error signal is produced. In some embodiments, modulating the one or more light sources comprises modulating illumination intensity.

In some embodiments, the system comprises one or more microtiter plate. For example, a 96-well plate or a 384-well plate can be used. In some embodiments, the system comprises a dispenser configured to dispense one or more reagents to the microtiter plate.

In some embodiments, the photoswitchable ion channel comprises a Synthetic Photoisomerizable Azobenzene-Regulated K+ (SPARK) channel or a variant thereof and the photoswitch regulator comprises Maleimide-Azobenzene-Quartenary ammonium (MAQ) or a variant thereof. In some embodiments, the photoswitchable ion channel comprises a glutamate receptor. In some embodiments, the photoswitchable ion channel comprises a Light-activated ionotropic Glutamate Receptor (LiGluR) channel or a variant thereof and the photoswitch regulator comprises Maleimide-Azobenzene-Glutamate (MAG) or a variant thereof. In some embodiments, the photoswitchable ion channel comprises a sodium channel. In some embodiments, the photoswitchable ion channel comprises an epithelial sodium channel (ENaC). In some embodiments, the photoswitch regulator for the sodium channel comprises a Maleimide-Azobenzene-Amiloride (MAA) or a variant thereof. In some embodiments, the system further comprises triamterene or amiloride to block the sodium channel.

In another aspect, the present invention provides an optical detection device comprising: one or more light sources; one or more dichroic beam combiners configurable to combine light from the one or more light sources; a sample lens array configurable to image combined light from the one or more dichroic beam combiners onto a sample array; and a detector lens array configurable to image light from the sample array onto a detector array.

In some embodiments, the one or more light sources comprise one or more source arrays. In some embodiments, the one or more light sources comprise two or more excitation sources, wherein each excitation source is optically coupled to separate optics.

In some embodiments, the one or more source arrays comprise light emitting diodes (LEDs). In some embodiments, the device further comprises a source lens array optically coupled to each source array. In some embodiments, the device further comprises one or more telescopes and/or one or more field lens. In some embodiments, the one or more telescopes are configurable to magnify or demagnify light passing from the sample array to the detector array. In some embodiments, the one or more source arrays are configurable to be arranged in the same pattern as a sample array or a portion thereof. In some embodiments, the sample lens array is configurable to collect fluorescence emitted from the samples. In some embodiments, the device further comprises a dichroic fluorescence beamsplitter configured to reflect light from the one or more dichroic beam combiners to the sample array, and transmit light passing from the sample array to the detector array. In some embodiments, the dichroic fluorescence beamsplitter is configurable to separate fluorescence from excitation radiation. In some embodiments, the one or more dichroic beam combiners and the sample lens array are configured on the opposite side of the sample array from the detector array. In some embodiments, the device further comprises a fluorescence collection lens array and a spatial filter array configurable to filter light passing from the sample array to the detector array. In some embodiments, the source arrays, the sample arrays and the detector arrays are on the same pitch. In some embodiments, the device comprises a dispenser configured to dispense one or more reagents to the sample array.

In another aspect, the present invention provides an optical detection device comprising: two or more excitation sources, wherein each excitation source is optically coupled to separate optics; one or more dichroic beam combiners configurable to combine light from the two or more excitation sources; a sample lens array configurable to image combined light from the one or more dichroic beam combiners onto a sample array; and a detector lens array configurable to image light from the sample array onto a detector array.

In some embodiments, the device further comprises one or more telescopes and/or one or more field lens In some embodiments, the one or more telescopes are configurable to magnify or demagnify light passing from the sample array to the detector array. In some embodiments, each excitation source emits light at a different wavelength. In some embodiments, the separate optics of the excitation sources comprise conventional flat beam lenses. In some embodiments, the separate optics of the excitation sources comprise microlens arrays. In some embodiments, the microlens arrays are configurable to produce flattop beams in circular or rectangular distributions. In some embodiments, the microlens arrays are configurable to produce rectangular arrays of individual beams. In some embodiments, the sample lens array is configurable to collect fluorescence emitted from the samples. In some embodiments, the device further comprises a dichroic fluorescence beamsplitter configured to reflect light from the one or more dichroic beam combiners to the sample array, and transmit light passing from the sample array to the detector array. In some embodiments, the dichroic fluorescence beamsplitter is configurable to separate fluorescence from excitation radiation. In some embodiments, the one or more dichroic beam combiners and the sample lens array are configured on opposite sides of the sample array from the detector array. In some embodiments, the device comprises a dispenser configured to dispense one or more reagents to the sample array.

In some embodiments of the method for determining the effect of a compound on a test channel, the optical measurement is performed using an optical detection device as described above. In some embodiments of the system for determining the effect of a compound on a test channel, the optical measurement is performed using an optical detection device configurable as described above.

In another aspect, the present invention provides kits. In some embodiments, a kit comprises one or more of materials, instructions, and devices configurable and adaptable to carry out the methods described herein. In some embodiments, a kit comprises one or more components of the systems described herein. In some embodiments, a kit comprises one or more optical detection devices as described herein.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a flow chart of an exemplary assay protocol of the invention.

FIGS. 2A-D illustrate several embodiments of the assay to determine the effect of a compound on a target test channel. FIG. 2A illustrates an assay wherein the compound blocks the target test channel in the open state. FIG. 2B illustrates an assay wherein the compound prevents the test channel from entering the inactivated state. FIG. 2C illustrates an assay as in FIG. 2A wherein two light sources are used to activate and inactive the photoswitchable ion channel and the compound blocks the test channel in the open state. FIG. 2D illustrates an assay as in FIG. 2B wherein two light sources are used to activate and inactive the photoswitchable ion channel, and the compound prevents the test channel from entering the inactivated state.

FIG. 3 illustrates an optical instrument for performing assays of the invention.

FIG. 4 illustrates an optical instrument as in FIG. 3 but without the telescope and field lens.

FIG. 5 illustrates a transmission geometry optical detection instrument for performing assays of the invention.

FIG. 6 illustrates an optical instrument for performing assays of the invention.

FIG. 7 illustrates an optical instrument as in FIG. 3, further comprising a dispenser.

### DETAILED DESCRIPTION OF THE INVENTION

The present invention provides a system for assaying ion-channels. In some embodiments, the system performs a plurality assays in parallel and in high-throughput. In some embodiments, the ion-channel assay provides a reversible change to the membrane potential of a target, e.g., a cell, upon exposure to light. The system further provides readout of the membrane potential, e.g., over a range of within a range of about −100 mV to +50 mV. In some embodiments, the readout is performed substantially in real-time. In some embodiments, the membrane potential readout is fed back through a control circuit to regulate the excitation intensity of the illumination sources that induce, respectively, hyperpolarizing and depolarizing currents, thereby effecting closed-loop control of the membrane potential.

I. Ion Channels

The two major families of ion channels comprise the voltage-gated ion channels and the ligand gated ion channels. Following are listed the major families of voltage-gated channels:

(a) Voltage-Gated Sodium Channels

This family is largely responsible for action potential creation and propagation. The pore-forming, α subunits are very large (up to 4,000 amino acids) and consist of four homologous repeat domains (I-IV) each comprising six transmembrane segments (S1-S6) for a total of 24 transmembrane segments. The members of this family also coassemble with auxiliary β subunits, each spanning the membrane once. A variety of different isoforms of mammalian voltage dependent sodium channels have been identified, and are summarized below in Table 1. These channels can be classified into three main groups (for review see Goldin, Annals N.Y. Academy of Sciences 868:38-50, 1999).

TABLE 1
Sodium Channel Sub-type Summary

| Channel Name & | Sub-type/ | Tissue | Accession |
|---|---|---|---|
| Gene Symbol | Alternate names | Distribution | Number |
| SCN1A (Nav1.1) | Rat I (rat) | CNS/PNS | X03638 |
| | HBSCI (human) | CNS | X65362 |
| | GPB1 (Guinea pig) | CNS | AF003372 |
| SCN2A (Nav1.2) | Rat II (rat) | CNS | X03639 |
| | HBSCII (human) | CNS | X65361 |
| | HBA (human) | CNS | M94055 |
| Nav1.2A | Rat IIA | CNS | X61149 |
| SCN3A (Nav1.3) | Rat III (rat) | CNS | Y00766 |
| SCN4A (Nav1.4) | SkM1, μ1 (rat) | Skeletal muscle | M26643 |
| | SkM1 (human) | Skeletal muscle | N81758 |
| SCN5A (Nav1.5) | SkM2 (rat) | Skeletal muscle/ | M27902 |
| | RH1(rat) | Heart | |
| | H1(human) | heart | M77235 |
| SCN8A (Nav1.6) | NaCh6 (rat) | CNS/PNS | L39018 |
| | PN4a (rat) | CNS/PNS | AF049239A |
| | Scn8a (mouse) | CNS | U26707 |
| | ScnSa (human) | CNS | AF050736 |
| | CerIII (Guinea pig) | CNS | AF003373 |
| SCN9A (Nav1.7) | PN1 (rat) | PNS | U79568 |
| | HNE-Na (human) | thyroid | X82835 |
| | Nas (rabbit) | Schwann cells | U35238 |
| SCN9A Nav1.7 | SNS (rat) | PNS | X92184 |
| | PN3 (rat) | PNS | U53833 |
| | SNS (mouse) | PNS | Y09108 |
| SCN6A Nav2.1 | Na2.1 (human) | Heart, uterus, | M91556 |
| | | muscle | |
| SCN7A Nav2.2 | Na-G (rat) | Astrocytes | M96578 |
| | SCL11(rat) | PNS | Y09165 |
| Nav2.3 | Na2.3 (mouse) | Heart, uterus, | L36179 |
| Nav3.1 | | muscle | |
| SCN1B Naβ1.1 | β-1 (rat) | CNS | M91808 |
| | β-1 (human) | CNS | L10338 |
| SCN2B Naβ2.1 | β-2 (rat) | CNS | U37026 |
| | β-2 (human) | CNS | AF007783 |

(b) Voltage-Gated Calcium Channels

This family plays an important role linking muscle excitation to contraction and neuronal excitation to transmitter release. The α subunits have an overall structural similarity to the sodium channels.

Among other functions, calcium channels play important roles in signal transduction. In excitable cells, intracellular calcium supplies a maintained inward current for long depolarizing responses and serves as the link between depolarization and other intracellular signal transduction mechanisms. Like voltage-gated sodium channels, voltage-gated calcium channels have multiple resting, activated, and inactivated states.

Multiple types of calcium channels have been identified in mammalian cells from various tissues, including skeletal muscle, cardiac muscle, lung, smooth muscle and brain, [see, e.g., Bean, B. P. (1989) Ann. Rev. Physiol. 51:367-384 and Hess, P. (1990) Ann. Rev. Neurosci. 56:337]. The different types of calcium channels have been broadly categorized into four classes, L-, T-, N-, and P-type, distinguished by current kinetics, holding potential sensitivity and sensitivity to calcium channel agonists and antagonists. Four subtypes of neuronal voltage-dependent calcium channels have been proposed (Swandulla, D. et al., Trends in Neuroscience 14:46, 1991).

(c) Voltage-Gated Potassium Channel

Voltage-gated potassium channels play a role in repolarizing the cell membrane following action potentials. The α subunits have six transmembrane segments, homologous to a single domain of the sodium channels. Correspondingly, they assemble as tetramers to produce a functioning channel.

Voltage-dependent potassium channels repolarize nerve and muscle cells after action potential depolarization. They also play important regulatory roles in neural, muscular, secretory, and excretory systems.

A summary of the numerous potassium sub-types is presented in Table 2 below.

TABLE 2
Potassium Channel Sub-type Summary

| | Sub-type/ | Accession | |
|---|---|---|---|
| Channel Type | Alternate names | Number | Reference |
| ATP regulated | rKir.1 (ROMK1) (rat) | U12541 | U.S. Pat. No. 5,356,775 |
| | hKirl.1 (ROMK1) (human) | | U.S. Pat. No. 5,882,873 |
| | Kirl.2 | U73191 | |
| | Kirl.3 | U73193 | |
| II. | Bcell | | U.S. Pat. No. 5,744,594 |
| III. | hβIR | | U.S. Pat. No. 5,917,027 |
| IV. | HuK_{ATP}-1 | | EP0 768 379A1 |
| Constitutively active | Kir2.1(IRK1) | U12507 | U.S. Pat. No. 5,492,825 |
| | | | U.S. Pat. No. 5,670,335 |
| | Kir2.2 | X78461 | |
| | Kir2.3 | X78461 | |
| G-protein regulated | Kir3.1 (GIK1, KGA) | U0171 | U.S. Pat. No. 5,728,535 |
| | Kir3.2 | U11859 | U.S. Pat. No. 5,734,021 |
| | Kir3.3 | U11869 | U.S. Pat. No. 5,744,324 |
| | Kir3.4 (CIR) | X83584 | U.S. Pat. No. 5,747,278 |
| | Kir4.1(BIR10) | X83585 | |
| | Kir5.1(BIR9) | X83581 | |
| | Kir6.1 | D42145 | |
| | Kir6.2 | D5081 | |
| | Kir7.1 | | EP0 922 763A1 |
| Voltage regulated | | | |
| KCNA1 | hKv1.1 (RCK1, RBK1, MBK1, | LO2750 | |
| | MK1, HuK1) | | |
| KCNA2 | hKv1.2 (RBK2, RBK5, NGK1, | | |
| | HuKIV) | | |
| KCNA3 | Kv1.3 (KV3, RGK5, HuKiIII, | | |
| | HPCN3) | | |
| KCNA4 | Kv1.4 (RCK4, RHK1, HuKII) | | |
| KCNA5 | Kv1.5 (KV1, HPCN1, HK2) | | |
| KCNA6 | Kv1.6 (KV2, RCK2, HBK2) | | |
| KCNA7 | Kv1.7 (MK6, RK6, HaK6) | | U.S. Pat. No. 5,559,009 |
| Kv2 (Shab) | | | |
| KCNB1 | Kv2.1 (DRK1, mShab) | M64228 | |
| KCNB2 | Kv2.2 (CDRK1) | | U.S. Pat. No. 5,710,019 |
| | K channel 2 | | |
| Kv3 (Shaw) | | | |
| KCNB1 | Kv3.1 (NGK2) | | |
| KCNB2 | Kv3.2 (KshIIIA) | | |
| KCNB3 | Kv3.3 (KshIIID) | X607796 | |
| KCNB4 | Kv3.4 (Raw3) | | |
| Kv4 (Sh1) | | | |
| KCND1 | Kv4.1 (mShal, KShIVA) | M64226 | |
| KCND2 | Kv4.2 (RK5, Rat Shal1) | | |
| KCND3 | Kv4.3 (KShIVB) | | WO 99/41372 |
| | hKv5.1(IK8) | | |
| | Kv6.1 (K13) | | |
| | Kv7 | | |
| | Kv8.1 | | |
| | Kv 9 | | |
| Delayed Rectifier | KvLQT1 | AF000571 | U.S. Pat. No. 5,599,673 |
| | HERG (erg) | U04270 | WO 99/20760 |
| Calcium regulated | | | |
| Calcium regulated Big | | | |
| | BKCa(hSLO) | U11717 | |
| | HBKb3 (β subunit) | | WO 99/42575 |
| | Maxi-K | | U.S. Pat. No. 5,776,734 |
| | | | U.S. Pat. No. 5,637,470 |
| Calcium regulated | | | |
| Calcium regulated Small | | | |
| KCNN1 | SKCa1 | U69883 | |
| KCNN2 | SKCa2 | U69882 | |
| KCNN3 | SKCa3 | U69884 | |
| KCNN4 | SKCa4 (IKCa1) | | Muscle Nerve 1999 |
| | | | 22(6) 742-50 |
| | TWIK1 | U33632 | |

(d) Voltage-Gated Chloride Channels

Chloride channels are found in the plasma membranes of virtually every cell in the body. Chloride channels mediate a variety of cellular functions including regulation of transmembrane potentials and absorption and secretion of ions across epithelial membranes. When present in intracellular membranes of the Golgi apparatus and endocytic vesicles, chloride channels also regulate organelle pH. For a review, see Greger, R. (1988) Annu. Rev. Physiol. 50:111-122.

Three distinct classes of chloride channels are apparent based on their type of regulation and structural conformation, Table 3. The first class includes the GABA and Glycine receptor super families, the second class includes the CFTR (Cystic fibrosis Transmembrane Conductance Regulator) and the third class includes the voltage regulated chloride channels.

TABLE 3
Chloride Channel Sub-type Summary

| | | Tissue | |
|---|---|---|---|
| Channel Type | Sub-type | Distribution | Reference |
| Ligand gated | GABA_{A} | CNS & PNS | Synapse 21, |
| | Receptor family | | 189-274 |
| | | | (1995) |
| | Glycine | CNS & PNS | Trends Neurosci. |
| | Receptor family | | 14 458-461 |
| | | | (1991) |
| cAMP regulated | CFTR | Epithelial | Science 245, |
| | | cells | 1066-1073 |
| | | | (1989) |
| Voltage | CIC-1 | Skeletal | Nature 354, |
| regulated | | muscle | 301-304 |
| | | | (1991) |
| | CIC-1 | Ubiquitous | Nature 356, |
| | | | 57-60 (1992) |
| | CIC-Ka | Kidney | J. Biol. Chem. 268, |
| | | | 3821-3824 (1993) |
| | CIC-Kb | Kidney | PNAS 91, |
| | | | 6943-6947 |
| | | | (1994) |
| | CIC-3 | Broad, e.g. | Neuron 12, |
| | | kidney & | 597-604 |
| | | brain | (1994) |
| | CIC-4 | Broad, e.g. | Hum. Nol. Genet. |
| | | kidney & | 3, 547-552 (1994) |
| | | brain | |
| | CIC-5 | Mainly | J. Biol. Chem. |
| | | kidney | 270, 31172- |
| | | | 31177 91995) |
| | CIC-6 | Ubiquitous | FEBS Lett. 377, |
| | | | 15-20 (1995) |
| | CIC-7 | Ubiquitous | FEBS Lett. 377, |
| | | | 15-20 (1995) |

(e) Transient Receptor Potential (TRP) Channels

These channels are diverse in the methods of activation. Some TRP channels are constitutively open; others are gated by voltage, Ca²⁺, pH, mechanical stretch, or other changes. These channels also vary according to the ion(s) they pass, some being selective for Ca²⁺; others are less selective cation channels. This family is subdivided into subfamilies based on homology, including classical (TRPC), vanilloid receptors (TRPV), melastatin (TRPM), polycystins (TRPP), mucolipins (TRPML), and ankyrin transmembrane protein 1 (TRPA).

(f) Hyperpolarization-Activated Cyclic Nucleotide-Gated Channels

These channels open in response to hyperpolarization rather than the depolarization and are also sensitive to the cyclic nucleotides cAMP and cGMP, which alter the voltage sensitivity of the channel opening. These channels are permeable to K⁺ and Na⁺. The family members form tetramers of six-transmembrane α subunits. These channels function as pacemaking channels in the heart, particularly the SA node.

(g) Voltage-Gated Proton Channels

These channels open with depolarization, in a strongly pH-sensitive manner, such that they open only when the proton electrochemical gradient is outward, allowing protons to leave the cell. In phagocytes (e.g. eosinophils, neutrophils, macrophages) during the respiratory burst that follows the ingestion of bacteria or other microbes, the enzyme NADPH oxidase assembles in the membrane and begins to produce reactive oxygen species (ROS) that help kill bacteria. NADPH oxidase is electrogenic, moving electrons across the membrane, and proton channels open to allow proton flux to balance the electron movement electrically.

(h) Ligand-Gated Ion Channels

Ligand gated ion channels, also known as ionotropic receptors, open in response to the binding of specific molecules to the extracellular domain of the receptor protein. Ligand binding causes a conformational change in the structure of the channel protein that ultimately leads to a change in the state of the channel gate and the consequent ion flux across the plasma membrane. Examples of such channels include the cation-permeable nicotinic acetylcholine receptor, ionotropic glutamate-gated receptors, ATP-gated P2X receptors, and the anion-permeable γ-aminobutyric acid-gated (GABAA) receptor. Ion channels activated by second messengers may also be categorized in this group. The response of many ligand-gated channels is voltage dependent.

The systems and methods of the present invention can be used to efficiently assay the effects of modulators on voltage-gated and ligand-gated ion channels. In some embodiments, these ion channels are co-expressed with light regulated, or photoswitchable, ion channels that can be used to controllably affect membrane potential. In some embodiments, modulating compounds, e.g., small molecules, peptides, proteins, nucleic acids, and the like, are screened for their effects on these ion channels.

II. Light-Regulated Ion Channels

Photoswitches are a family of small-molecules configurable to modulate the function of specific signaling proteins, e.g., ion-channels, thereby enabling control of the proteins with light: one color turns the protein ‘on,’ a different color turns the protein ‘off.’ The photoswitch comprises a plurality of functional components: (1) a ligand, that binds reversibly to an active site on an ion channel or other protein; (2) an optional anchoring moiety that binds, e.g., covalently, to an motif or a single residue, e.g., near the active site of the ion channel or other protein; and (3) a light-sensitive moiety that changes configuration upon absorption of light in a particular range of frequencies and which resumes its initial configuration upon absorption of light of second range of frequencies. Azobenzene is an example of a light-sensitive moiety. Azobenzene isomerizes from the trans to the cis conformation in the presence of illumination in the violet-near-ultraviolet and switches from the cis form back to the trans conformation, either spontaneously (which can take from a few ms to 10 s of minutes, depending on the substituents) or in the presence of illumination in the blue-green (which requires ˜1 ms).

Several classes of photoswitch molecules have been developed. One type of photoswitch molecule covalently attaches to a residue engineered into the target protein. See, e.g., Banghart, M., Borges, K., Isacoff, E., Trauner, D. & Kramer, R. H., Light-activated ion channels for remote control of neuronal firing, Nature Neuroscience 7, 1381-1386 (2004). For example, Maleimide-Azobenzene-Quartenary ammonium (MAQ), is composed of tetraethylammonium (TEA), a ligand for K⁺ channels, azobenzene, and a maleimide moiety that covalently attaches to a cysteine residue engineered into the target ion channel protein. This type of photoswitch comprises the use of mutant ion channels, such as the Synthetic Photoisomerizable Azobenzene-Regulated K⁺ (SPARK) channel. SPARK channels are shaker-type K⁺ channels that have been genetically modified to remove inactivation. Both hyperpolarizing (h-SPARK) and depolarizing (d-SPARK) light activated channels have been reported. The photoswitch MAQ is a small rigid molecule that changes length by about 7 Å upon photoisomerization. The pore of the channel is blocked when MAQ is in the trans configuration. 380-nm light converts the photoswitch to the cis form, relieving TEA-induced block and allowing K⁺ ions to flow. 500-nm light reverses the process, re-blocking current flow through the channel. Other engineered, light-sensitive ion channels have been developed including an ionotropic glutamate receptor, the Light-activated ionotropic Glutamate Receptor (LiGluR). See, e.g., Gorostiza, P., Volgraf, M., Numano, R., Szobota, S., Trauner, D. and Isacoff, E. Y., Mechanisms of photoswitch conjugation and light activation of an ionotropic glutamate receptor, PNAS 104, 10865-70 (2007). Photocontrol of LiGluR is based on the reversible photoisomerization of Maleimide-Azobenzene-Glutamate (MAG). MAG is covalently attached by the maleimide moiety to a cysteine introduced into the ligand-binding domain of the receptor. Photoswitching of the ionotropic current is driven by the reversible binding of the glutamate moiety, which is presented to the ligand-binding site in the cis configuration and withdrawn in trans. Under 380-nm illumination, the glutamate moiety of the MAG activates the receptor and opens its cation-selective channel, resulting in membrane depolarization.

Another type of photoswitch, called Photoaffinity Labels, substitute a promiscuous reactive group, acrylamide, for the cysteine-specific maleimide, on one end of the photoswitch molecule. The acrylamide can form a covalent attachment with nucleophiles present in the native ion channel protein. See, e.g., Fortin, et al., 2008 [Fortin D L, Banghart M R, Dunn T W, Borges K, Wagenaar D A, Gaudry Q, Karakossian M H, Otis T S, Kristan W B, Trauner D and Kramer R H, Photochemical control of endogenous ion channels and cellular excitability, Nature Methods 5, 331-8 (2008). For example, Acrylamide-Azobenzene-Quaternary ammonium (AAQ) forms a covalent bond between the acrylamide group on the photoswitch and an endogenous nucleophile group on the channel. In the trans configuration, the photoswitch molecule is elongated and the channel blocking Quaternary Ammonium (QA) moiety can reach the channel mouth where it binds, preventing the passage of K⁺ ions through the channel pore. When the photoswitch is in the cis form, the QA group is unable to reach the binding site in the channel mouth and the channel allows the passage of K⁺ ions.

A third class of photoswitch, exemplified by Benzyl-Azobenzene-Quaternary ammonium (BzAQ), imparts light sensitivity on K⁺ channels in the absence of covalent attachment. These untethered molecules, termed Photochromic Ligands, are similar to AAQ but possess an unreactive acetamide at one end of the molecule, instead of acrylamide. Complete photoswitching of wild-type Shaker K⁺ channels treated with this compound has been observed. Light sensitivity can persist for hours after washing, suggesting that the compound has partitioned into the membrane or has otherwise formed a stable association with the membrane and/or ion channel protein. Ion flux through this K⁺ channel can be regulated by light such that when BzAQ is in the elongated trans configuration the QA moiety has access to the internal QA blocking site. Illumination with 380-nm light causes the photoisomerization of the molecule into the cis configuration, such that the QA ligand is unable to access the blocking site allowing K⁺ ions to flow through the channel.

Certain photoswitches of the present invention effectively latch in the two states. For example, the rate of thermal relaxation, from cis to trans, in the dark for MAG in solution can be about 18 mins. Continuous illumination is not necessarily required to maintain photoswitched ion channels in open or closed states.

The present invention provides photoswitch-controlled ion channels, also referred to as photoswitched ion channels. Any class of photoswitch molecules can be used, e.g., photoswitch molecules that covalently bind mutant ion channels, photoswitch molecules that covalently bind to native ion channels, or photoswitch molecules that do not covalently bind the corresponding ion channel. Photoswitchable ion channels are described in further detail below.

III. Photoswitch-Enabled Ion Channel Assays

The present invention provides high throughput ion channel assays using photoswitchable ion channels to rapidly, repeatedly, and uniformly change and control membrane potential. In some embodiments, the assay is performed by illuminating some or all of a multi-well plate comprising, e.g., 96, 384, or 1536 wells. The uniformity of membrane potential changes across the cells in the wells of a multiwall plate allows the use of an averaged response, measured by, e.g., fast voltage-sensitive dyes, voltage-sensitive FRET dyes, ion-sensitive dyes, semiconductor nanocrystals, or by the induced field from the illuminated cells in each well. The assay can be performed without large objectives, complicated imaging hardware and software, or expensive, precision sample translation stages.

(a) Assay

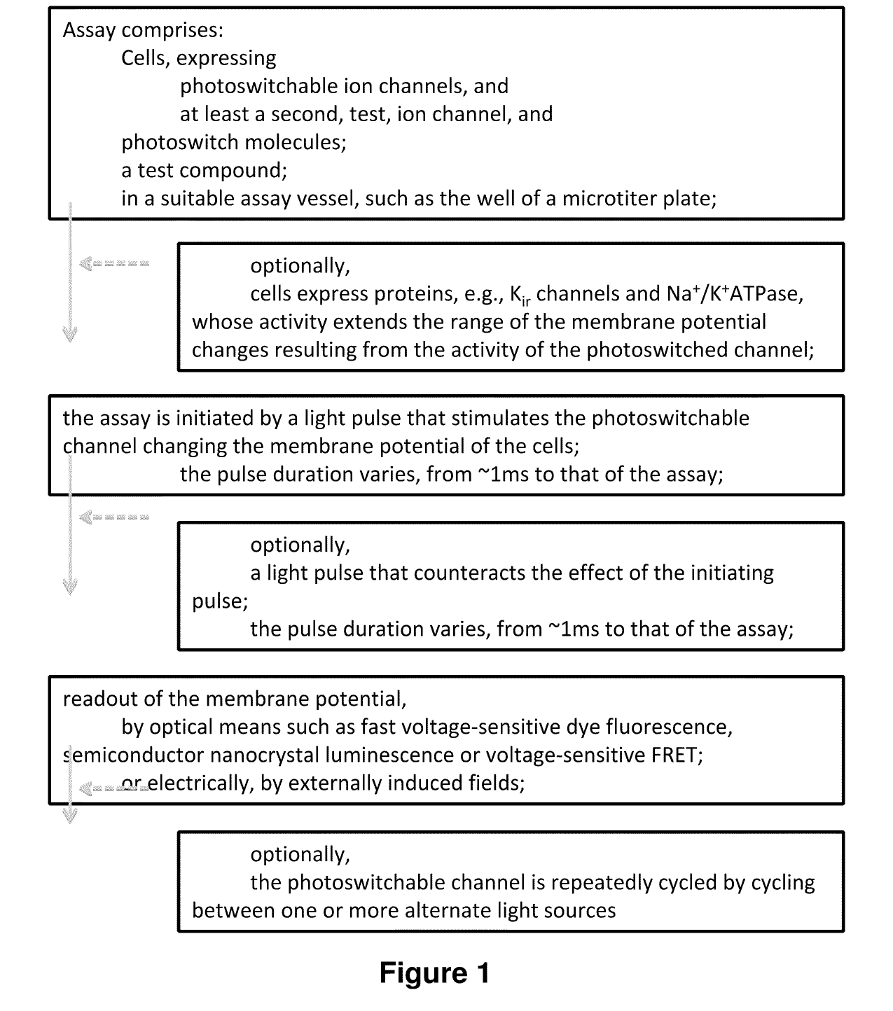

In one aspect, the invention provides a method for determining the effect of a compound on a test channel. The method comprises the steps of: a) providing cells expressing both the test channel and a photoswitchable ion channel; b) contacting the cells with a photoswitch regulator of the photoswitchable ion channel; c) illuminating the contacted cells with a light source that modulates the photoswitchable ion channel; and d) determining a membrane potential of the cells, thereby determining the effect of the compound on the test channel. A general schematic of the assay is shown in FIG. 1.

The method can be used to identify drug candidates in a high throughput manner by performing the assay on a large number of compounds to identify those that modulate the activity of the test channel. In some embodiments, the compounds are an agonist, an antagonist, an allosteric modulator, or a blocker of the test channel. Modulation can include both upregulation, (i.e., activation or stimulation) for example by agonizing, and downregulation (i.e., inhibition or suppression) for example by antagonizing, a test channel as measured by assessing a biological parameter (e.g., membrane potential). An inhibitor or agonist may cause partial or complete modulation of activity. By modulating the activity of the test channel, the compound can produce a physiological effect on the cell. Some compounds identified by the methods of the invention can act as therapeutic agents. Therapeutic agents include molecules or atoms which are useful for therapy. Examples of therapeutic agents include drugs, toxins, immunomodulators, chelators, antibodies, antibody-drug conjugates, photoactive agents or dyes, and radioisotopes.

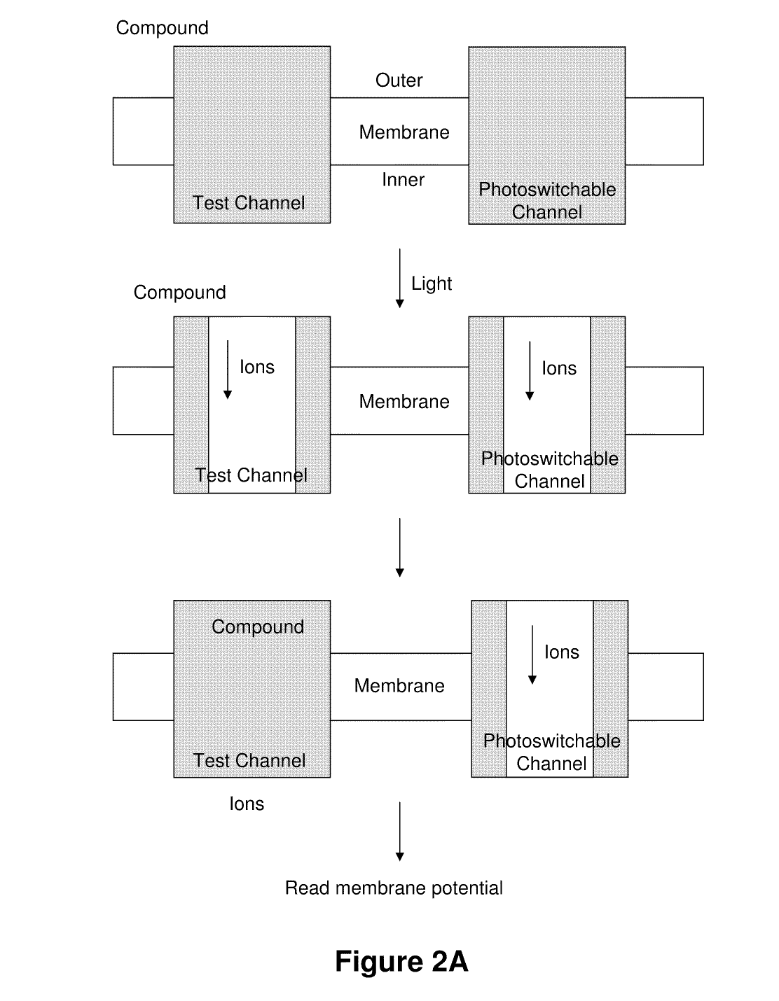

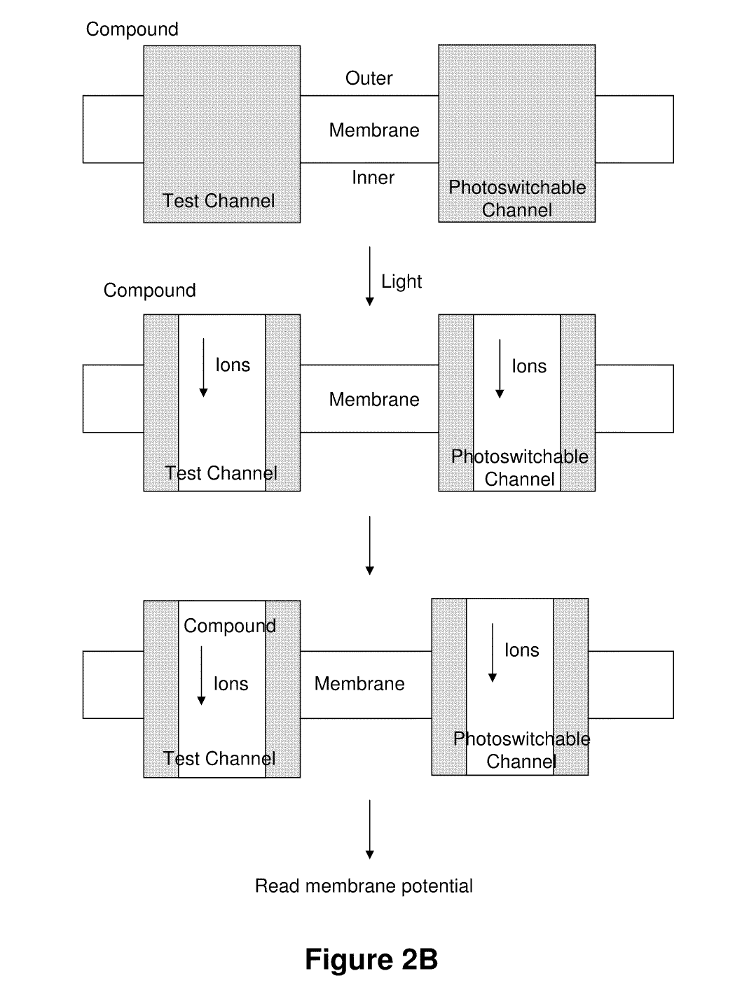

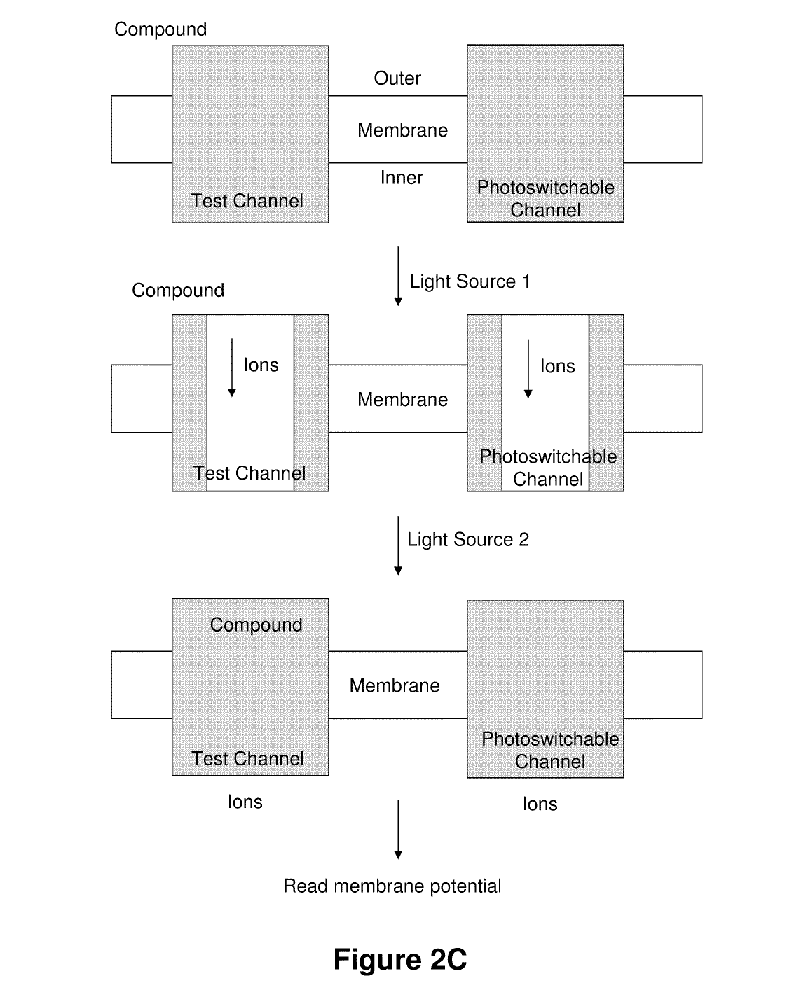

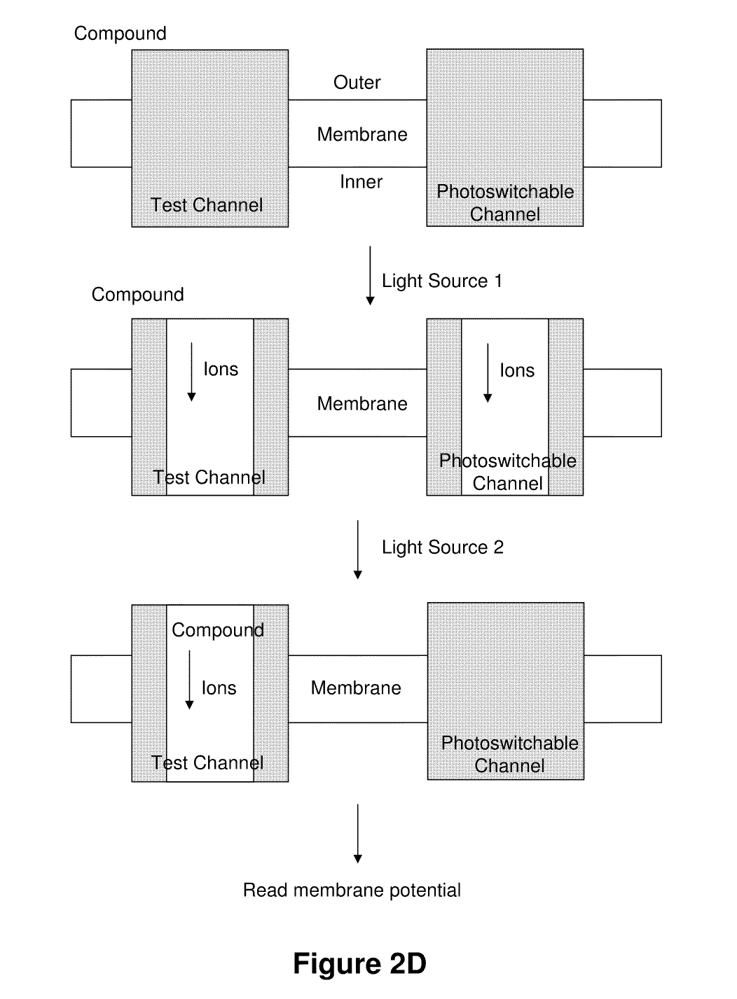

Illustrative examples of the assay are illustrated in FIG. 2. In FIG. 2A, the target test channel and the photoswitchable channel are initially closed. Upon exposure to light, the photoswitchable channel is activated, allowing ions to enter the cell. In this exemplary embodiment, the test channel opens in response to the change in membrane potential resulting from the opening of the photoswitched channel. The test compound blocks the test channel in the open state. In FIG. 2B, the target test channel and the photoswitchable channel are initially closed. Upon exposure to light, the photoswitchable channel is activated, allowing ions to enter the cell. In this exemplary embodiment, the test channel opens in response to the change in membrane potential resulting from the opening of the photoswitched channel. The compound prevents the test channel from entering the inactivated state. In both exemplary embodiments of FIGS. 2A-B, the membrane potential is read to determine the effect of the compound. In some embodiments, the membrane potential is read continuously throughout the assay. One of skill in the art will appreciate that any number of combinations are possible with the methods of the present invention. For example, the compound can activate, overactivate, inactivate or block the test channel. Similarly, the photoswitch can activate, overactivate, inactivate or block the photoswitchable ion channel. Any of these effects can be partial or complete.

In some embodiments, the compound comprises a small-molecule, e.g., a drug compound, a polypeptide, or a nucleic acid. Polypeptides, including peptides and proteins, include polymeric forms of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. Polypeptides can comprise one or more of a fatty acid moiety, a lipid moiety, a sugar moiety, and a carbohydrate moiety. In some cases, polypeptides include post-translational modifications.

Nucleic acid or nucleic acid molecules include polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules also include peptide nucleic acids (PNA), which comprise naturally-occurring or modified nucleic acid bases attached to a polyamide backbone. Nucleic acids can be either single stranded or double stranded.

In some embodiments, the test channel is exogenous, i.e., not native, to the cell. For example, the test channel may be introduced by recombinant DNA or similar technologies. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In other embodiments, the test channel is native to the cell. For example, a cell can be chosen that naturally expresses the desired test channel. In some embodiments, the cell is influenced to overexpress the native test channel, e.g., by supplementing the growth media with certain agents.

In some embodiments, the test channel is a voltage-dependent ion channel. In some embodiments, the voltage-dependent ion channel is a voltage-gated ion channel or a ligand-gated ion channel, as described herein. The invention therefore provides methods and systems to identify compounds that modulate ion channels, to provide candidate therapeutic agents.

The assay can be modulated by the illumination source. In some embodiments, illumination of the photoswitchable ion channel by the light source stimulates, activates, inactivates or blocks the channel. These effects can be partial or complete. The time of illumination can affect the modulation of the photoswitchable ion channel. For example, cells that are illuminated for a shorter time period may have less modulation that those illuminated for a longer time period.

Two or more light sources can be used to modulate the photoswitchable ion channel. In some embodiments, the methods of the invention comprise illuminating the cells with a second light source after illuminating the cells with the first light source. In some embodiments, the illumination by the second light source counteracts the modulation of the photoswitchable ion channel by the first light source. For example, if the first light source causes a conformation change in the photoswitch that activates the photoswitchable ion channel, a second light source may cause another conformation change in the photoswitch that deactivates the photoswitchable ion channel. In some embodiments, the method further comprises repeatedly alternating illumination of the cells with the first and second light sources. In some embodiments, the cells are periodically exposed to multiple light sources in a sequential manner, an overlapping manner, or any combination thereof. In these embodiments, the membrane potential of the cells can be monitored throughout the assay, or can be readout at the end of the illumination protocol. Such cycling can have a variety of effects, e.g., changing the membrane potential between two or more values, increasing the fraction of test channels in a state of interest, or increasing the effect of the test compound on the test channel.

Illustrative examples of the assay with two light sources are illustrated in FIG. 2. In FIG. 2C, the target test channel and the photoswitchable channel are initially closed. Upon exposure to light, the photoswitchable channel is activated, allowing ions to enter the cell. The corresponding change in membrane potential opens the test channel. A second light source is then used to inactivate or block the photoswitchable channel. In this exemplary embodiment, the compound blocks the targeted test channel in the open state. In FIG. 2D, the target test channel and the photoswitchable channel are initially closed. Upon exposure to light, the photoswitchable channel is activated, allowing ions to enter the cell. The test channel opens as the membrane potential exceeds its threshold potential. In this exemplary embodiment, the compound prevents the open test channel from entering the inactivated state, and the channel remains open after the illumination from the second light source has caused the photoswitched channel to close. In these embodiments, the membrane potential is read to determine the effect of the compound. In some embodiments, the membrane potential is read continuously. One of skill in the art will appreciate that any number of combinations are possible with the methods of the present invention. For example, the compound can activate, overactivate, inactivate or block the test channel. Similarly, the photoswitch can activate, overactivate, inactivate or block the photoswitchable ion channel. Any of these effects can be partial or complete.

(b) Photoswitch Regulators

The present invention makes use of photoswitch regulators of protein function. Examples of synthetic photoswitch regulators have been described in U.S. patent application Ser. No. 11/601,591, filed Nov. 17, 2006 and entitled “Photoreactive regulator of protein function and methods of use thereof”; U.S. patent application Ser. No. 12/388,800, filed Dec. 18, 2008 and entitled “Photoreactive regulator of glutamate receptor function and methods of use thereof”; U.S. Provisional Patent Application No. 61/110,369, filed Oct. 31, 2008 and entitled “Photoreactive regulator of protein function and methods of use thereof”; and U.S. Provisional Patent Application No. 61/122,608, filed Dec. 15, 2008 and entitled “Photoreactive regulator of protein function and methods of use thereof,” all of which applications are incorporated by reference herein in their entirety. Such synthetic regulators of protein function are useful for regulating protein function by use of light. In some embodiments, a synthetic protein regulator comprises: a) a protein association moiety, which can provide for interaction with the polypeptide; b) a photoisomerizable group; and c) a ligand that binds to a ligand binding site (e.g., an active site, an allosteric site, a pore of an ion channel, etc.) of a protein. A synthetic protein regulator (also referred to herein as a “synthetic regulator,” “photoswitch regulator,” or “a photoswitch”) is suitable for attachment to a variety of polypeptides, including naturally-occurring (native, or endogenous) polypeptides, recombinant polypeptides, synthetic polypeptides, etc.

A photoswitch regulator can be provided in any number of configurations, including linear and branched. In some embodiments, a photoswitch regulator has the structure: (A)ₙ-X₁—(B)ₘ—X₂—(C)ₚ, where A is an optional protein association moiety, B is a photoisomerizable group, and C is a ligand, and where each of n, m, and p is independently 1 to 10, e.g., where each of n, m, and p is independently one, two, three, four, five, six, seven, eight, nine, or ten, and where X₁, when present, is a spacer; and X₂, when present, is a spacer. In some embodiments, X₁ and X₂ are not present. In some embodiments, X₁ and X₂ are both present. In some embodiments, only one of X₁ and X₂ is present. In some embodiments, X₁ and X₂ are independently selected from alkyl, substituted alkyl, alkenyl, substituted alkenyl, alkynyl, substituted alkynyl, acyl, acylamino, and aminoacyl. In some embodiments, each of n, m, and p is 1. In other embodiments, a photoswitch regulator comprises two or more (e.g., 2 to 10, e.g., two, three, four, five, six, seven, eight, nine, or ten) photoisomerizable groups. In some embodiments, where the synthetic regulator comprises two or more photoisomerizable groups, the two or more photoisomerizable groups are arranged in tandem, either directly or separated by a spacer.

A photoswitch regulator can be provided in any number of configurations, including linear and branched. In some embodiments, a photoswitch regulator has the structure: (A)ₙ-(B)ₘ—(C)ₚ, where A is a protein association moiety, B is a photoisomerizable group, and C is a ligand, and where each of n, m, and p is independently 1 to 10, e.g., where each of n, m, and p is independently one, two, three, four, five, six, seven, eight, nine, or ten. In some embodiments, each of n, m, and p is 1, e.g., a photoswitch regulator has the structure A-B-C.

In other embodiments, a photoswitch regulator has the structure: C—X₁(A)-B-X₂(A)-C, where A is a protein association moiety, B is a photoisomerizable group, and C is a ligand, where X₁, when present, is a spacer, where X₂, when present, is a spacer, and where X(A) indicates that A branches off of X. Suitable spacers include peptide spacers (e.g., spacers of from about 1 to about 20 amino acids in length); non-peptide spacers, e.g., non-peptide polymers of various numbers of monomeric units, e.g., from one to about 20 units. In these embodiments, B can be present in multiple copies, either directly or in tandem.

(i) Protein Association Moiety

The protein association moiety can be any of a variety of functional groups that provide for association of the synthetic regulator with a polypeptide. In some embodiments, the protein association moiety can provide for association with an amino acid side chain in a polypeptide. In some embodiments, the protein association moiety can provide for association with a ligand-binding polypeptide, and with a membrane component. In other embodiments, the protein association moiety can provide for association of the synthetic regulator with a sugar residue in the polypeptide. In other embodiments, the protein association moiety can provide for association of the synthetic regulator with a moiety other than a sugar residue or an amino acid side chain. In some embodiments, the protein association moiety can comprise a reactive electrophile that can provide for association with an amino acid in the ligand-binding polypeptide. In some embodiments, the protein association moiety can comprise a reactive electrophile that can provide for association with an amino acid at or near a ligand-binding site in a ligand-binding protein.

Association of the synthetic regulator with a polypeptide includes non-covalent associations such as ionic interactions, van der Waals interactions, hydrogen bonding, and the like. The association is a high-affinity association, e.g., the association between the synthetic regulator and the polypeptide has an affinity of from about 10⁻³M to about 10⁻¹² M, or greater than 10⁻¹²M, e.g, the association between the synthetic regulator and the polypeptide has an affinity of from about 10⁻³ M to about 5×10⁻³ M, from about 5×10⁻³ M to about 10⁻⁴ M, from about 10⁻⁴ M to about 5×10⁻⁴M, from about 5×10⁻⁴ M to about 10⁻⁵ M, from about 10⁻⁵M to about 5×10⁻⁵ M, from about 5×10⁻⁵ M to about 10⁻⁶ M, from about 10⁻⁶ M to about 5×10⁻⁶ M to about 10⁻⁷ M, from about 10⁻⁷ M to about 5×10⁻⁷ M, from about 5×10⁻⁷ M to about 10⁻⁸M, from about 10⁻⁸ M to about 5×10⁻⁸M, from about 5×10⁻⁸M to about 10⁻⁹M, from about 10⁻⁹ M to about 5×10⁻⁹ M, from about 5×10⁻⁹ M to about 10⁻¹⁰ M, from about 10⁻¹⁰ M to about 5×10⁻¹⁰ M, from about 5×10⁻¹⁰ M to about 10⁻¹¹ M, from about 5×10⁻¹¹ M to about 10⁻¹² M, or greater. In some embodiments, e.g., where a photoswitch regulator comprises two or more protein association moieties, each of the moieties can provide for attachment to a polypeptide with an affinity of less than about 10⁻⁹ M, but together the two or more protein association moieties provide for a binding affinity that is 10⁻⁹ M or greater. In some embodiments, e.g., where a photoswitch regulator comprises two or more protein association moieties, each of the moieties can provide for attachment to a polypeptide with an affinity of less than about 10⁻⁴ M, but together the two or more protein association moieties provide for a binding affinity that is 10⁻⁴ M or greater.

In certain embodiments, the protein association moiety comprises a group selected from hydrogen, C₁-C₁₀ alkyl, substituted C₁-C₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, C(O)OR¹⁰, —SR¹⁰, —S(O)R¹⁰, —S(O)₂R¹⁰; wherein

R¹⁰ and R¹¹ are independently selected from hydrogen and C₁-C₁₀ alkyl;

R¹² is hydrogen or C₁-C₁₀ alkyl;

R¹³ is selected from hydrogen, C₁-C₁₀ alkyl, C₁-C₈ alkenyl, C₆-C₁₀ aryl, and substituted C₁-C₁₀ alkyl.

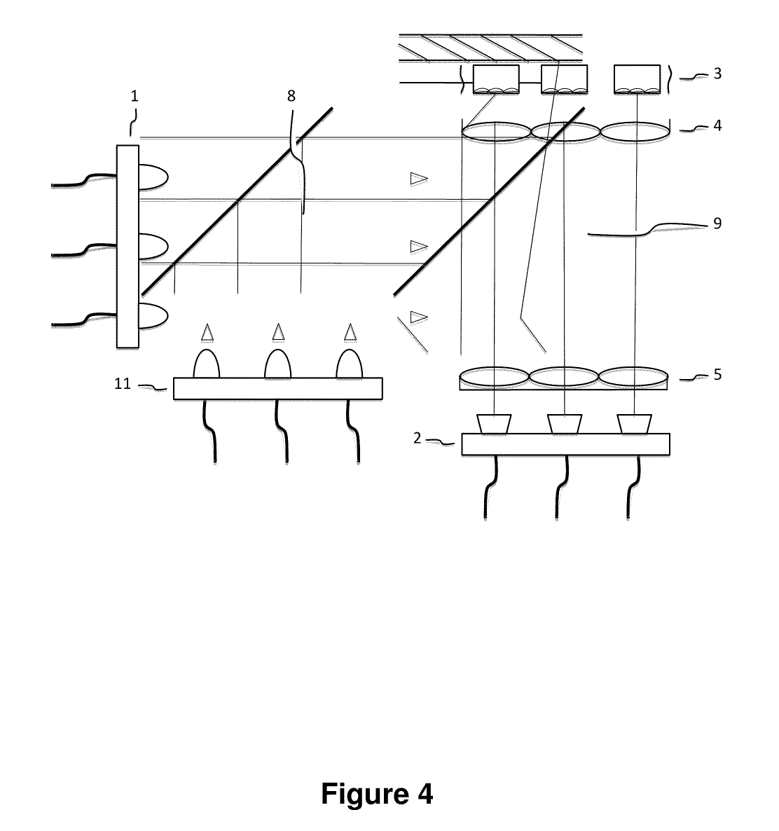

Exemplary suitable protein association moieties are depicted in FIG. 4. In FIG. 4, protein association moieties are designated “R.”

(ii) Photoisomerizable Group

The photoisomerizable group is one that changes from a first isomeric form to a second isomeric form upon exposure to light of different wavelengths, or upon a change in exposure from dark to light, or from light to dark. For example, in some embodiments, the photoisomerizable group is in a first isomeric form when exposed to light of a first wavelength, and is in a second isomeric form when exposed to light of a second wavelength. Suitable photoisomerizable groups include stereoisomers and constitutional isomers.

The first wavelength and the second wavelength can differ from one another by from about 1 nm to about 2000 nm or more, e.g., from about 1 nm to about 10 nm, from about 10 nm to about 20 nm, from about 20 nm to about 50 nm, from about 50 nm to about 75 nm, from about 75 nm to about 100 nm, from about 100 nm to about 125 nm, from about 125 nm to about 150 nm, or from about 150 nm to about 200 nm, from about 200 nm to about 500 nm, from about 500 nm to about 800 nm, from about 800 nm to about 1000 nm, from about 1000 nm to about 1500 nm, from about 1500 nm to about 2000 nm, or more than 2000 nm.

In other embodiments, the photoisomerizable group is in a first isomeric form when exposed to light of a wavelength λ₁, and is in a second isomeric form in the absence of light (e.g., in the absence of light, the photoisomerizable group undergoes spontaneous relaxation into the second isomeric form). In these embodiments, the first isomeric form is induced by exposure to light of wavelength λ₁, and the second isomeric form is induced by not exposing the photoisomerizable group to light, e.g., keeping the photoisomerizable group in darkness. In other embodiments, the photoisomerizable group is in a first isomeric form in the absence of light, e.g., when the photoisomerizable group is in the dark; and the photoisomerizable group is in a second isomeric form when exposed to light of a wavelength λ₁. In other embodiments, the photoisomerizable group is in a first isomeric form when exposed to light of a first wavelength λ₁, and the photoisomerizable group is in a second isomeric form when exposed to light of second wavelength λ₂.

For example, in some embodiments, the photoisomerizable group is in a trans configuration in the absence of light, or when exposed to light of a first wavelength; and the photoisomerizable group is in a cis configuration when exposed to light, or when exposed to light of a second wavelength that is different from the first wavelength. As another example, in some embodiments, the photoisomerizable group is in a cis configuration in the absence of light, or when exposed to light of a first wavelength; and the photoisomerizable group is in a trans configuration when exposed to light, or when exposed to light of a second wavelength that is different from the first wavelength.

The wavelength of light that effects a change from a first isomeric form to a second isomeric form ranges from 10⁻⁸ m to about 1 m, e.g., from about 10⁻⁸ m to about 10⁻⁷ m, from about 10⁻⁷ m to about 10⁻⁶ m, from about 10⁻⁶ m to about 10⁻⁴ m, from about 10⁻⁴ m to about 10⁻² m, or from about 10⁻² m to about 1 m. “Light,” as used herein, refers to electromagnetic radiation, including, but not limited to, ultraviolet light, visible light, infrared, and microwave.

The wavelength of light that effects a change from a first isomeric form to a second isomeric form ranges in some embodiments from about 200 nm to about 800 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, or from about 750 nm to about 800 nm, or greater than 800 nm.

In other embodiments, the wavelength of light that effects a change from a first isomeric form to a second isomeric form ranges from about 800 nm to about 2500 nm, e.g., from about 800 nm to about 900 nm, from about 900 nm to about 1000 nm, from about 1000 nm to about 1200 nm, from about 1200 nm to about 1400 nm, from about 1400 nm to about 1600 nm, from about 1600 nm to about 1800 nm, from about 1800 nm to about 2000 nm, from about 2000 nm to about 2250 nm, or from about 2250 nm to about 2500 nm. In other embodiments, the wavelength of light that effects a change from a first isomeric form to a second isomeric form ranges from about 2 nm to about 200 nm, e.g., from about 2 nm to about 5 nm, from about 5 nm to about 10 nm, from about 10 nm to about 25 nm, from about 25 nm to about 50 nm, from about 50 nm to about 75 nm, from about 100 nm, from about 100 nm to about 150 nm, or from about 150 nm to about 200 nm.

The difference between the first wavelength and the second wavelength can range from about 1 nm to about 2000 nm or more, as described above. Of course, where the synthetic light regulator is switched from darkness to light, the difference in wavelength is from essentially zero to a second wavelength.

The intensity of the light can vary from about 1 W/m² to about 50 W/m², e.g., from about 1 W/m² to about 5 W/m², from about 5 W/m² to about 10 W/m², from about 10 W/m², from about 10 W/m² to about 15 W/m², from about 15 W/m² to about 20 W/m², from about 20 W/m² to about 30 W/m², from about 30 W/m² to about 40 W/m², or from about 40 W/m² to about 50 W/m². The intensity of the light can vary from about 1 μW/cm² to about 100 μW/cm², e.g., from about 1 μW/cm² to about 5 μW/cm², from about 5 μW/cm² to about 10 μW/cm², from about 10 μW/cm² to about 20 μW/cm², from about 20 μW/cm² to about 25 μW/cm², from about 25 μW/cm² to about 50 μW/cm², from about 50 μW/cm² to about 75 μW/cm², or from about 75 μW/cm² to about 100 μW/cm². In some embodiments, the intensity of light varies from about 1 μW/mm² to about 1 W/mm², e.g., from about 1 μW/mm² to about 50 μW/mm², from about 50 μW/mm² to about 100 μW/mm², from about 100 μW/mm² to about 500 μW/mm², from about 500 μW/mm² to about 1 mW/mm², from about 1 mW/mm² to about 250 mW/mm², from about 250 mW/mm² to about 500 mW/mm², or from about 500 mW/mm² to about 1 W/mm². In some embodiments, the intensity of the light is about 5 W/m², or 5 mW/mm².

In some embodiments, the change from a first isomeric form to a second isomeric form of the photoisomerizable group is effected using sound, instead of electromagnetic (EM) radiation (light). For example, in some embodiments, the change from a first isomeric form to a second isomeric form of the photoisomerizable group is effected using ultrasound.

Photoisomerizable groups are known in the art, and any known photoisomerizable group can be included in a photoswitch regulator of protein function. Suitable photoisomerizable groups include, but are not limited to, azobenzene and derivatives thereof; spiropyran and derivatives thereof; triphenyl methane and derivatives thereof; 4,5-epoxy-2-cyclopentene and derivatives thereof; fulgide and derivatives thereof; thioindigo and derivatives thereof; diarylethene and derivatives thereof; diallylethene and derivatives thereof; overcrowded alkenes and derivatives thereof; and anthracene and derivatives thereof. In some embodiments, a suitable photoisomerizable group is a photoisomerizable group as shown in the examples herein.

Suitable spiropyran derivatives include, but are not limited to, 1,3,3-trimethylindolinobenzopyrylospiran; 1,3,3-trimethylindolino-6′-nitrobenzopyrylospiran; 1,3,3-trimethylindolino-6′-bromobenzopyrylospiran; 1-n-decyl-3,3-dimethylindolino-6′-nitrobenzopyrylospiran; 1-n-octadecy-1-3,3-dimethylindolino-6′-nitrobenzopyrylospiran; 3′,3′-dimethyl-6-nitro-1′-[2-(phenylcarbamoyl)ethyl]spiro; [2H-1-benzopyran-2,2′-indoline]; 1,3,3-trimethylindolino-8′-methoxybenzopyrylospiran; and 1,3,3-trimethylindolino-β-naphthopyrylospiran. Also suitable for use is a merocyanine form corresponding to spiropyran or a spiropyran derivative.

Suitable triphenylmethane derivatives include, but are not limited to, malachite green derivatives. specifically, there can be mentioned, for example, bis[dimethylamino)phenyl]phenylmethanol, bis[4-(diethylamino)phenyl]phenylmethanol, bis[4-(dibuthylamino)phenyl]phenylmethanol and bis[4-(diethylamino)phenyl]phenylmethane.

Suitable 4,5-epoxy-2-cyclopentene derivatives include, for example, 2,3-diphenyl-1-indenone oxide and 2′,3′-dimethyl-2,3-diphenyl-1-indenone oxide.

Suitable azobenzene compounds include, e.g., compounds having azobenzene residues crosslinked to a side chain, e.g., compounds in which 4-carboxyazobenzene is ester bonded to the hydroxyl group of polyvinyl alcohol or 4-carboxyazobenzene is amide bonded to the amino group of polyallylamine. Also suitable are azobenzene compounds having azobenzene residues in the main chain, for example, those formed by ester bonding bis(4-hydroxyphenyl)dimethylmethane (also referred to as bisphenol A) and 4,4′-dicarboxyazobenzene or by ester bonding ethylene glycol and 4,4′-dicarboxyazobenzene.

Suitable fulgide derivatives include, but are not limited to, isopropylidene fulgide and adamantylidene fulgide.

Suitable diallylethene derivatives include, for example, 1,2-dicyano-1,2-bis(2,3,5-trimethyl-4-thienyl)ethane; 2,3-bis(2,3,5-trimethyl-4-thiethyl)maleic anhydride; 1,2-dicyano-1,2-bis(2,3,5-trimethyl-4-selenyl)ethane; 2,3-bis(2,3,5-trimethyl-4-selenyl)maleic anhydride; and 1,2-dicyano-1,2-bis(2-methyl-3-N-methylindole)ethane.

Suitable diarylethene derivatives include but are not limited to, substituted perfluorocylopentene-bis-3-thienyls and bis-3-thienylmaleimides.

Suitable overcrowded alkenes include, but are not limited to, cis-2-nitro-7-(dimethylamino)-9-(2′,3′-dihydro-1′H-naphtho[2,1-b]thiopyran-1′-ylidene)-9H-thioxanthene and trans-dimethyl-[1-(2-nitro-thioxanthen-9-ylidene)-2,3-dihydro-1H-benzo[f]thiochromen-8-yl]amine. Overcrowded alkenes are described in the literature. See, e.g., terWiel et al. (2005) Org. Biomol. Chem. 3:28-30; and Geertsema et al. (1999) Agnew CHem. Int. Ed. Engl. 38:2738.

Other suitable photoisomerizable moieties include, e.g., reactive groups commonly used in affinity labeling, including diazoketones, aryl azides, diazerenes, and benzophenones.

(iii) Ligands

As used herein, the term “ligand” refers to a molecule (e.g., a small molecule, a peptide, or a protein) that binds to a polypeptide and effects a change in an activity of the polypeptide, and/or effects a change in conformation of the polypeptide, and/or affects binding of another polypeptide to the polypeptide. Ligands include agonists, partial agonists, inverse agonists, antagonists, allosteric modulators, and blockers.

In some embodiments, the ligand is a naturally-occurring ligand. In other embodiments, the ligand is a synthetic ligand. In other embodiments, the ligand is an endogenous ligand. In some embodiments, the ligand is an agonist. In other embodiments, the ligand is an inverse agonist. In other embodiments, the ligand is a partial agonist. In other embodiments, the ligand is an antagonist. In other embodiments, the ligand is an allosteric modulator. In other embodiments, the ligand is a blocker. An antagonist generally refers to an agent that binds to a ligand-binding polypeptide and inhibits an activity of the ligand-binding polypeptide. An antagonist may be an agent that binds to an allosteric site but does not activate the ligand-binding polypeptide; instead, the antagonist generally excludes binding by an agonist and thus prevents or hinders activation. A blocker can include an agent that acts directly on the active site, pore, or allosteric site. Ligands suitable for use herein bind reversibly to a ligand-binding site of a ligand-binding polypeptide.

The ligand is selected based in part on the activity of the polypeptide to which the synthetic regulator will be attached. For example, a ligand for a hormone-binding transcription factor is a hormone, or a synthetic analog of the hormone. A ligand for a tetracycline transactivator is tetracycline or a synthetic analog thereof. A ligand for an enzyme will in some embodiments be a synthetic agonist or antagonist of the enzyme. In some embodiments, a ligand will block the ligand-binding site. A ligand for a ligand-gated ion channel will in some embodiments be a naturally-occurring ligand, or a synthetic version of the ligand, e.g., a synthetic analog of the ligand. In some embodiments, the ligand is other than an acetylcholine receptor ligand. In some embodiments, the ligand is other than trimethylammonium.

In some embodiments, a ligand is a small molecule ligand. Small molecule ligands generally have a molecular weight in a range of from about 50 daltons to about 3000 daltons, e.g., from about 50 daltons to about 75 daltons, from about 75 daltons to about 100 daltons, from about 100 daltons to about 250 daltons, from about 250 daltons to about 500 daltons, from about 500 daltons to about 750 daltons, from about 750 daltons to about 1000 daltons, from about 1000 daltons to about 1250 daltons, from about 1250 daltons to about 1500 daltons, from about 1500 daltons to about 2000 daltons, from about 2000 daltons to about 2500 daltons, or from about 2500 daltons to about 3000 daltons.

In other embodiments, a ligand is a peptide ligand. Peptide ligands can have a molecular weight in a range of from about 1 kDa to about 20 kDa, e.g., from about 1 kDa to about 2 kDa, from about 2 kDa to about 5 kDa, from about 5 kDa to about 7 kDa, from about 7 kDa to about 10 kDa, from about 10 kDa to about 12 kDa, from about 12 kDa to about 15 kDa, or from about 15 kDa to about 20 kDa.

Suitable ligands include, but are not limited to, ligands that block or activate the function of a ligand-binding protein, where ligand-binding proteins include channels; receptors (including, but not limited to, ionotropic receptors that bind transmitters; ionotropic receptors that bind hormones; metabotropic receptors; receptor tyrosine kinases; growth factor receptors; and other membrane receptors that signal by binding to soluble or membrane-bound or extracellular matrix-bound small molecules or proteins); transporters (including but not limited to ion transporters, organic molecule transporters, peptide transporters, and protein transporters); enzymes (including but not limited to kinases; phosphatases; ubiquitin ligases; acetylases; oxo-reductases; lipases; enzymes that add lipid moieties to proteins or remove them; proteases; and enzymes that modify nucleic acids, including but not limited to ligases, helicases, topoisomerases, and telomerases); motor proteins (including kinesins, dyenins and other microtobule-based motors, myosins and other actin-based motors, DNA and RNA polymerases and other motors that track along polynucleotides); scaffolding proteins; adaptor proteins; cytoskeletal proteins; and other proteins that localize or organize protein domains and superstructures within cells.

Suitable ligands include, but are not limited to, ligands that function as general anesthetics; ligands that function as local anesthetics; ligands that function as analgesics; synthetic and semi-synthetic opioid analgesics (e.g., phenanthrenes, phenylheptylamines, phenylpiperidines, morphinans, and benzomorphans) where exemplary opioid analgesics include morphine, oxycodone, fentanyl, pentazocine, hydromorphone, meperidine, methadone, levorphanol, oxymorphone, levallorphan, codeine, dihydrocodeine, hydrocodone, propoxyphene, nalmefene, nalorphine, naloxone, naltrexone, buprenorphine, butorphanol, nalbuphine, and pentazocine; ionotropic glutamate receptor agonists and antagonists, e.g., N-methyl-D-aspartate (NMDA) receptor agonists and antagonists, kainate (KA) receptor agonists and antagonists, and α-amino-3-hydroxy-5-methylisoxazole-4-propionic acid (AMPA) receptor agonists and antagonists; non-opioid analgesics, e.g., acetylsalicylic acid, choline magnesium trisalicylate, acetaminophen, ibuprofen, fenoprofen, diflusinal, and naproxen; muscarinic receptor agonists; muscarinic receptor antagonists; acetylcholine receptor agonists; acetylcholine receptor antagonists; serotonin receptor agonists; serotonin receptor antagonists; enzyme inhibitors; a benzodiazepine, e.g. chlordiazepoxide, clorazepate, diazepam, flurazepam, lorazepam, oxazepam, temazepam or triazolam; a barbiturate sedative, e.g. amobarbital, aprobarbital, butabarbital, butabital, mephobarbital, metharbital, methohexital, pentobarbital, phenobartital, secobarbital, talbutal, theamylal, or thiopental; an H₁ antagonist having a sedative action, e.g. diphenhydramine, pyrilamine, promethazine, chlorpheniramine, or chlorcyclizine; an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, topiramate, neramexane, or perzinfotel; an alpha-adrenergic, e.g. doxazosin, tamsulosin, clonidine, guanfacine, dexmetatomidine, modafinil, phentolamine, terazasin, prazasin or 4-amino-6,7-dimethoxy-2-(5-methane-sulfonamido-1,2,3,4-tetrahydroisoquinol-2-yl)-5-(2-pyridyl)quinazoline; a tricyclic antidepressant, e.g. desipramine, imipramine, amitriptyline, or nortriptyline; an anticonvulsant, e.g. carbamazepine, lamotrigine, topiratmate, or valproate; a tachykinin (NK) antagonist, particularly an NK-3, NK-2 or NK-1 antagonist, e.g. (α-R,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S); a muscarinic antagonist, e.g oxybutynin, tolterodine, propiverine, tropsium chloride, darifenacin, solifenacin, temiverine, or ipratropium; a cyclooxygenase-2 (COX-2) selective inhibitor, e.g. celecoxib, rofecoxib, parecoxib, valdecoxib, deracoxib, etoricoxib, or lumiracoxib; a vanilloid receptor agonist (e.g. resinferatoxin) or antagonist (e.g. capsazepine); a beta-adrenergic such as propranolol; a 5-HT receptor agonist or antagonist, e.g., a 5-HT₁B/₁D agonist such as eletriptan, sumatriptan, naratriptan, zolmitriptan or rizatriptan; a 5-HT₂A receptor antagonist such as R(+)-α-(2,3-dimethoxy-phenyl)-1-[2-(4-fluorophenylethyl)]-4-piperidinemethanol (MDL-100907); and the like.

Suitable ligands for Na⁺ channels include, but are not limited to, lidocaine, novocaine, xylocaine, lignocaine, novocaine, carbocaine, etidocaine, procaine, prontocaine, prilocaine, bupivacaine, cinchocaine, mepivacaine, quinidine, flecainide, procaine, N-[[2′-(aminosulfonyl)biphenyl-4-yl]methyl]-N′-(2,2′-bithien-5-ylmethyl)succinamide (BPBTS), QX-314, saxitoxin, tetrodotoxin, and a type III conotoxin. Suitable ligands for Na⁺ channels also include, but are not limited to, tetrodotoxin, saxitoxin, guanidinium, polyamines (e.g. spermine, cadaverine, putrescine, μ-conotoxin, and δ-conotoxin.

Suitable ligands for K⁺ channels include, but are not limited to, quaternary ammonium (e.g., tetraethyl ammonium, tetrabutylammonium, tetrapentylammonium), 4-aminopyridine, sulfonylurea, Glibenclamide; Tolbutamide; Phentolamine, qiunine, qunidine, peptide toxins (e.g., charybdotoxin, agitoxin-2, apamin, dendrotoxin, VSTX1, hanatoxin-1, hanatoxin-2, and Tityus toxin K-α.

Suitable ligands for CNG and HCN channels include, but are not limited to, 1-cis diltiazem and ZD7288. Suitable ligands for glycine receptors include, but are not limited to, strychnine and picrotoxin.

Suitable ligands for nicotinic acetylcholine receptors include, but are not limited to, (+)tubocurarine, Methyllycaconitine, gallamine, Nicotine; Anatoxin A, epibatidine, ABT-94, Lophotoxin, Cytisine, Hexamethonium, Mecamylamine, and Dihydro-β-erythroidine. Suitable ligands for muscarinic acetylcholine receptors include, but are not limited to, a muscarinic acetylcholine receptor antagonist as described in U.S. Pat. No. 7,439,255; AF267B (see, e.g., U.S. Pat. No. 7,439,251); phenylpropargyloxy-1,2,5-thiadiazole-quinuclidine; carbachol; pirenzapine; migrastatin; a compound as described in U.S. Pat. No. 7,232,841; etc.

Suitable ligands for GABA receptors include, but are not limited to, Muscimol, THIP, Procabide, bicuculine, picrotoxin, gabazine, gabapentin, diazepam, clonazepam, flumazenil, a β-carboline carboxylate ethyl ester, baclofen, faclofen, and a barbiturate.

Many suitable ligands will be known to those skilled in the art; and the choice of ligand will depend, in part, on the target (e.g., receptor, ion channel, enzyme, etc.) to which the ligand binds.

In some embodiments, a photoswitchable ion channel of the present invention comprises epithelial sodium channel (ENaC; sodium channel non-neuronal 1 (SCNN1); amiloride sensitive sodium channel (ASSC)). ENaC is a membrane-bound, constitutively active Na⁺ selective ion-channel. It is one of the most selective channels known, being weakly permeable to Li+ and protons, in addition to Na+. In some embodiments, the channel is modified for use with a tethered photoswitch, e.g., Maleimide-Azobenzene-Amiloride (MAA). In some embodiments, the ENaC is blocked by triamterene or amiloride. In some embodiments, the photoswitch blocks in the extended, trans, conformation. An antagonist can be withdrawn from the binding site when the photoswitch is excited into the cis conformation. In some embodiments, an alternate configuration with cis blocking and trans activation is used.

(c) Photoswitchable Channels

The present invention takes advantage of numerous photoswitchable ion channels. In some embodiment, the invention uses mutant ion channels, such as the Synthetic Photoisomerizable Azobenzene-Regulated K⁺ (SPARK) channel. SPARK channels are shaker-type K⁺ channel that have been genetically modified to remove inactivation. Both hyperpolarizing (h-SPARK) and depolarizing (d-SPARK) light activated channels have been reported. In some embodiments, the photoswitch MAQ is used with SPARK channels. MAQ is a small rigid molecule that changes length by about 7 Å upon photoisomerization. The pore of the channel is blocked when MAQ is in the trans configuration. 380-nm light converts the photoswitch to the cis form, relieving QA-induced block and allowing K⁺ ions to flow. 500-nm light reverses the process, re-blocking current flow through the channel. In some embodiments the invention uses other engineered, light-sensitive ion channels, e.g, Light-activated ionotropic Glutamate Receptor (LiGluR), an ionotropic glutamate receptor. Photocontrol of LiGluR is based on the reversible photoisomerization of Maleimide-Azobenzene-Glutamate (MAG). MAG is covalently attached by the maleimide moiety to a cysteine introduced into the ligand-binding domain of the receptor. Photoswitching of the ionotropic current is driven by the reversible binding of the glutamate moiety, which is presented to the ligand-binding site in the cis configuration and withdrawn in trans. MAG binding under 380-nm light activates the receptor and opens its cation-selective channel, resulting in membrane depolarization.

The present invention makes use of light-regulated polypeptides, where a light-regulated polypeptide comprises a polypeptide and a photoswitch regulator of receptor function in association with the polypeptide. The synthetic regulator of polypeptide function can be either covalently or non-covalently associated with the polypeptide at or near a ligand binding site of the polypeptide. In some embodiments, a light-regulated polypeptide is isolated, e.g., free of other polypeptides or other macromolecules. In other embodiments, a light-regulated polypeptide is membrane-associated and is present in vitro. In other embodiments, a light-regulated polypeptide is present in a living cell in vitro or in vivo. In other embodiments, a light-regulated polypeptide is present in a membrane of a living cell in vitro or in vivo. In other embodiments, a light-regulated polypeptide is present in a living cell in a tissue in vitro or in vivo. In other embodiments, a light-regulated polypeptide is present in a living cell in a multicellular organism. Polypeptides with which a photoswitch regulator can be non-covalently associated include, e.g., receptors, ion channels, enzymes, and the like.

A change in the wavelength and/or intensity of light (Δλ) to which the light-regulated polypeptide is exposed results in a change in ligand binding to a ligand-binding site of the light-regulated polypeptide, e.g., results in a change in binding of the ligand portion of the synthetic polypeptide to the ligand-binding site of the light-regulated polypeptide. A “change in the wavelength of light to which the light-regulated polypeptide is exposed” includes: 1) a change from λ₁ to λ₂; 2) a change from λ₂ to λ₁; 3) a change from λ₁ to darkness (no light); and 4) a change from darkness to λ₁. Repetitive changing from λ₁ to λ₂, then from λ₂ to λ₁, and back, e.g., switching from a first wavelength to a second wavelength, and back again repeatedly, is also contemplated. Repetitive changing from light to darkness, from darkness to light, etc., is also contemplated.

In some embodiments, the change in wavelength (from λ₁ to λ₂; from light to darkness; or from darkness to light) results in a change in binding of the ligand to a ligand-binding site. As used herein, a “change in binding of a ligand to a ligand-binding site” includes increased binding and decreased binding. As used herein, “increased binding” includes one or more of: an increased probability of binding of the ligand to the ligand-binding site; an increased binding affinity of the ligand for the ligand-binding site; an increased local concentration of the ligand at the ligand-binding site; and an increased occupancy of the ligand in the ligand-binding site. As used herein, “decreased binding” includes one or more of: a decreased probability of binding of the ligand to the ligand-binding site; a decreased binding affinity of the ligand for the ligand-binding site; a decreased local concentration of the ligand at the ligand-binding site; and a decreased occupancy of the ligand in the ligand-binding site. As used herein, the term “change in wavelength” to which a synthetic regulator is exposed, or to which a receptor/synthetic light regulator complex is exposed, refers to a change in wavelength from λ₁ to λ₂; a change from light to darkness; or a change from darkness to light. An increase in binding includes an increase of from about 10% to about 50%, from about 50% to about 2-fold, from about 2-fold to about 5-fold, from about 5-fold to about 10-fold, from about 10-fold to about 50-fold, from about 50-fold to about 10²-fold, from about 10²-fold to about 10⁴-fold, from about 10⁴-fold to about 10⁶-fold, from about 10⁶-fold to about 10⁸-fold, or a greater than 10⁸-fold increase in binding. A decrease in binding includes a decrease of from about 5% to about 10% to about 20% to about 30%, from about 30% to about 40%, from about 40% to about 50%, from about 50% to about 60%, from about 60% to about 70%, from about 70% to about 80%, from about 80% to about 90%, or from about 90% to 100% decrease in binding.

For example, in some embodiments, the ligand has a first probability of binding to the ligand site at a first wavelength of light; the ligand has a second probability of binding to the ligand binding site at a second wavelength of light; and the second probability is lower than the first probability. In other embodiments, the ligand has a first probability of binding to the ligand site at a first wavelength of light; the ligand has a second probability of binding to the ligand binding site at a second wavelength of light; and the second probability is higher than the first probability. In other embodiments, ligand has a first probability of binding to the ligand site when exposed to light; the ligand has a second probability of binding to the ligand binding site in the absence of light (e.g., in darkness); and the second probability is lower than the first probability. In other embodiments, the ligand has a first probability of binding to the ligand site when exposed to light; the ligand has a second probability of binding to the ligand binding site in the absence of light and the second probability is higher than the first probability.

The local concentration of the ligand portion of the synthetic regulator at the ligand binding site in a light-regulated polypeptide is high. For example, the local concentration of the ligand portion of the synthetic regulator at the ligand binding site in a light-regulated polypeptide ranges from about 500 nM to about 50 mM, e.g., from about 500 nM to about 750 nM, from about 750 nM to about 1 mM, from about 1 mM to about 5 mM, from about 5 mM to about 10 mM, from about 10 mM to about 20 mM, from about 20 mM to about 30 mM, or from about 30 mM to about 50 mM.

In some embodiments, a change in the wavelength of light to which the light-regulated polypeptide is exposed results in an increase in binding affinity of the ligand portion of a photoswitch regulator for a ligand-binding site of the polypeptide portion of the light-regulated polypeptide. For example, in some embodiments, a change in wavelength of light to which the light-regulated polypeptide is exposed results in an at least about 10%, at least about 20%, at least about 30%, at least about 50%, at least about 75%, at least about 2-fold, at least about 5-fold, at least about 10-fold, at least about 25-fold, at least about 50-fold, at least about 100-fold, at least about 250-fold, at least about 500-fold, at least about 10³-fold, at least about 5×10³-fold, at least about 10⁴-fold, at least about 5×10⁴-fold, or greater, increase in binding affinity.

Where the ligand is an agonist, the change in wavelength will in some embodiments result in activation of the light-regulated polypeptide. Where the ligand is an agonist, the change in wavelength will in some embodiments result in desensitization of the light-regulated polypeptide. Conversely, where the ligand is an antagonist, the change in wavelength results in a block of activation of the light-regulated polypeptide, e.g., block of the ability to activate the light-regulated polypeptide with free agonist. Where the ligand is a blocker (e.g., a pore blocker of an ion channel, or an interaction domain that binds to other biological macromolecules such as polypeptides or nucleic acids), the change in wavelength results in block of polypeptide activity.

Expressed another way, where the ligand is an agonist, and where a change in the wavelength of light to which the light-regulated polypeptide is exposed results in a higher binding affinity of the ligand moiety of the synthetic regulator to the ligand-binding site of the light-regulated polypeptide, the change in wavelength results in transition from an inactive state to an active state, or to a desensitized state. Where the ligand is an antagonist, the change in wavelength results in transition from a responsive state to an unresponsive state. Where the ligand is a blocker, the change in wavelength results in transition from an active state to an inactive state.

In some embodiments, a change in the wavelength of light to which the light-regulated polypeptide is exposed results in removal of the ligand portion of a photoswitch regulator from a ligand-binding site of the light-regulated polypeptide, e.g., the ligand is not bound to the ligand-binding site. In some embodiments, a change in the wavelength of light to which the light-regulated polypeptide is exposed results in reduced binding affinity of the ligand portion of a photoswitch regulator for a ligand-binding site of the light-regulated polypeptide, e.g., the ligand has reduced binding affinity for the ligand-binding site. For example, in some embodiments, a change in the wavelength of light to which the light-regulated polypeptide is exposed results in a reduction of binding affinity of at least about 10%, at least about 20%, at least about 25%, at least about 30%, at least about 40%, at least about 50%, at least about 60%, at least about 70%, at least about 80%, at least about 90%, at least about 95%, or more.

Where the ligand is an agonist, the change in wavelength will in some embodiments result in deactivation of the light-regulated polypeptide. Where the ligand is an agonist, the change in wavelength will in some embodiments result in recovery from desensitization of the light-regulated polypeptide. Conversely, where the ligand is an antagonist, the change in wavelength results in activation of the light-regulated polypeptide, or results in removal of a blocker from the light-regulated polypeptide. Where the ligand is a blocker (e.g., a pore blocker of an ion channel, or an interaction domain that binds to other biological macromolecules such as polypeptides or nucleic acids), the change in wavelength results in relief of a block in polypeptide activity and permits the receptor to function normally.

Expressed another way, where the ligand is an agonist, and where a change in the wavelength of light to which the light-regulated polypeptide is exposed results in removal (or non-binding) of the ligand moiety of the synthetic regulator from the ligand-binding site of the light-regulated polypeptide, the change in wavelength results in transition from an active state to an inactive state, or from a desensitized state to a responsive state. Where the ligand is an antagonist, the change in wavelength results in transition from an unresponsive state to a responsive state. Where the ligand is a blocker, the change in wavelength results in transition from an inactive state to an active state.

In some embodiments, the polypeptide portion of a light-regulated polypeptide is an ion channel. A light-regulated ion channel comprises an ion channel and a photoswitch regulator of receptor function in association with the ion channel. The synthetic regulator of polypeptide function is covalently or non-covalently associated with the ion channel at or near a ligand binding site of the receptor on the ion channel. In some embodiments, the synthetic regulator can provide occlusion to the ion channel. Ion channels with which a photoswitch regulator of polypeptide function can be non-covalently associated include, e.g., sodium channels, potassium channels, calcium channels, and chloride channels. The ion channel can be voltage regulated, cAMP regulated, or ligand gated.

(d) Cells

The embodiments further provide a cell comprising a light-regulated polypeptide. Where the cell is used in a screening assay, the cell can be referred to as a test cell.

The cell can be an in vivo or in vitro prokaryotic cell, an in vivo or in vitro eukaryotic cell, or a cell from a multicellular organism (e.g., a cell line) cultured in vitro as a unicellular entity. A cell includes a cell that comprises a subject light-regulated polypeptide. A host cell includes cells that can be, or have been, used as recipients for a photoswitch regulator. Host cells includes cells that can be, or have been, used as recipients for an exogenous nucleic acid. It is understood that the progeny of a single cell may not necessarily be completely identical in morphology or in genomic or total DNA complement as the original parent, due to natural, accidental, or deliberate mutation. A recombinant host cell (also referred to as a “genetically modified host cell”) is a host cell into which has been introduced a heterologous nucleic acid, e.g., an expression vector. For example, in some embodiments a subject host cell is a genetically modified eukaryotic host cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

In some embodiments, the cell is a eukaryotic cell in in vitro cell culture, and is grown as an adherent monolayer, or in suspension. In other embodiments, the cell is a eukaryotic cell and is part of a tissue or organ, either in vivo or in vitro. In other embodiments, the cell is a eukaryotic cell and is part of a living multicellular organism, e.g., a protozoan, an amphibian, a reptile, a plant, an avian organism, a mammal, a fungus, an algae, a yeast, a marine microorganism, a marine invertebrate, an arthropod, an isopod, an insect, an arachnid, etc. In other embodiments, the cell is a prokaryotic cell.

In other embodiments, the cell is a member of archaea, e.g., an archaebacterium. Archaebacteria include a methanogen, an extreme halophile, an extreme thermophile, and the like. Suitable archaebacteria include, but are not limited to, any member of the groups Crenarchaeota (e.g., Sulfolobus solfataricus, Defulfurococcus mobilis, Pyrodictium occultum, Thermofilum pendens, Thermoproteus tenax), Euryarchaeota (e.g., Thermococcus celer, Methanococcus thermolithotrophicus, Methanococcus jannaschii, Methanobacterium thermoautotrophicum, Methanobacterium formicicum, Methanothermus fervidus, Archaeoglobus fulgidus, Thermoplasma acidophilum, Haloferax volcanni, Methanosarcina barkeri, Methanosaeta concilli, Methanospririllum hungatei, Methanomicrobium mobile), and Korarchaeota.

In some embodiments, the cell is of a particular tissue or cell type. For example, where the organism is a plant, the cell is part of the xylem, the phloem, the cambium layer, leaves, roots, etc. Where the organism is an animal, the cell will in some embodiments be from a particular tissue (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

A cell is in many embodiments a unicellular organism, or is grown in culture as a single cell suspension, or as monolayer. In some embodiments, a cell is a eukaryotic cell. Suitable eukaryotic cells include, but are not limited to, yeast cells, insect cells, plant cells, fungal cells, mammalian cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK293 cells, HLHepG2 cells, and the like.

In some embodiments, the cell is a neuronal cell or a neuronal-like cell. The cells can be of human, non-human primate, mouse, or rat origin, or derived from a mammal other than a human, non-human primate, rat, or mouse. In some embodiments, the neuronal cell is a primary cell isolated from an animal. In some embodiments, the neuronal cell or neuronal-liked cell is an immortalized cell line. Suitable cell lines include, but are not limited to, a human glioma cell line, e.g., SVGp12 (ATCC CRL-8621), CCF-STTG1 (ATCC CRL-1718), SW 1088 (ATCC HTB-12), SW 1783 (ATCC HTB-13), LLN-18 (ATCC CRL-2610), LNZTA3WT4 (ATCC CRL-11543), LNZTA3WT11 (ATCC CRL-11544), U-138 MG (ATCC HTB-16), U-87 MG (ATCC HTB-14), H4 (ATCC HTB-148), and LN-229 (ATCC CRL-2611); a human medulloblastoma-derived cell line, e.g., D342 Med (ATCC HTB-187), Daoy (ATCC HTB-186), D283 Med (ATCC HTB-185); a human tumor-derived neuronal-like cell, e.g., PFSK-1 (ATCC CRL-2060), SK-N-DZ (ATCCCRL-2149), SK-N-AS (ATCC CRL-2137), SK-N-FI (ATCC CRL-2142), IMR-32 (ATCC CCL-127), etc.; a mouse neuronal cell line, e.g., BC3H1 (ATCC CRL-1443), EOC1 (ATCC CRL-2467), C8-D30 (ATCC CRL-2534), C8-S (ATCC CRL-2535), Neuro-2a (ATCC CCL-131), NB41A3 (ATCC CCL-147), SW10 (ATCC CRL-2766), NG108-15 (ATCC HB-12317); a rat neuronal cell line, e.g., PC-12 (ATCC CRL-1721), CTX TNA2 (ATCC CRL-2006), C6 (ATCC CCL-107), F98 (ATCC CRL-2397), RG2 (ATCC CRL-2433), B35 (ATCC CRL-2754), R3 (ATCC CRL-2764), SCP (ATCC CRL-1700), OA1 (ATCC CRL-6538).

In other embodiments, the host cell is a plant cell. Plant cells include cells of monocotyledons (“monocots”) and dicotyledons (“dicots”). Guidance with respect to plant tissue culture may be found in, for example: Plant Cell and Tissue Culture, 1994, Vasil and Thorpe Eds., Kluwer Academic Publishers; and in: Plant Cell Culture Protocols (Methods in Molecular Biology 111), 1999, Hall Eds, Humana Press.

Suitable prokaryotic cells include bacteria (e.g., Eubacteria) and archaebacteria. Suitable archaebacteria include a methanogen, an extreme halophile, an extreme thermophile, and the like. Suitable archaebacteria include, but are not limited to, any member of the groups Crenarchaeota (e.g., Sulfolobus solfataricus, Defulfurococcus mobilis, Pyrodictium occultum, Thermofilum pendens, Thermoproteus tenax), Euryarchaeota (e.g., Thermococcus celer, Methanococcus thermolithotrophicus, Methanococcus jannaschii, Methanobacterium thermoautotrophicum, Methanobacterium formicicum, Methanothermus fervidus, Archaeoglobus fulgidus, Thermoplasma acidophilum, Haloferax volcanni, Methanosarcina barkeri, Methanosaeta concilli, Methanospririllum hungatei, Methanomicrobium mobile), and Korarchaeota. Suitable eubacteria include, but are not limited to, any member of Hydrogenobacteria, Thermotogales, Green nonsulfphur bacteria, Denococcus Group, Cyanobacteria, Purple bacteria, Planctomyces, Spirochetes, Green Sulphur bacteria, Cytophagas, and Gram positive bacteria (e.g., Mycobacterium sp., Micrococcus sp., Streptomyces sp., Lactobacillus sp., Helicobacterium sp., Clostridium sp., Mycoplasma sp., Bacillus sp., etc.).

Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the embodiments include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the cell is Escherichia coli.

(e) Membranes

The embodiments further provide a membrane comprising a light-regulated polypeptide. In some embodiments, the membrane is a biological membrane (e.g., a lipid bilayer surrounding a biological compartment such as a cell, including artificial cells, or a membrane vesicle or sheet). In some embodiments, the membrane is part of a living cell, as described above. In other embodiments, the membrane is an artificial (synthetic) membrane, e.g., a planar membrane, a liposome, etc.

In some embodiments, the artificial membrane is a lipid bilayer. In other embodiments, the artificial membrane is a lipid monolayer. In some embodiments, the artificial membrane is part of a liposome. Liposomes include unilamellar vesicles composed of a single membrane or lipid bilayer, and multilamellar vesicles (MLVs) composed of many concentric membranes (or lipid bilayers).

Artificial membranes, and methods of making same, have been described in the art. See, e.g., U.S. Pat. No. 6,861,260; Kansy et al. (1998) J. Med. Chem. 41(7):1007-10; and Yang et al. (1996) Advanced Drug Delivery Reviews 23:229-256.

A artificial membrane will in some embodiments, include one or more phospholipids. In some embodiments, the artificial membrane comprises a mixture of phospholipids containing saturated or unsaturated mono or disubstituted fatty acids and a combination thereof. These phospholipids are in some embodiments selected from dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, and palmiticlinoleoylphosphatidic acid. Suitable phospholipids also include the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in such lysophosphatidyl derivatives will in some embodiments be palimtoyl, oleoyl, palmitoleoyl, linoleoyl myristoyl or myristoleoyl.

(f) Modulating Photoswitchable Ion Channels

The embodiments of the present invention modulate protein activity. Some embodiments use methods of modulating activity of a light-regulated polypeptide, where the method generally involves changing the wavelength of light to which the light-regulated polypeptide is exposed. In certain aspects, the embodiments provide methods of modulating activity of a polypeptide, where the method generally involves: a) contacting the polypeptide with a photoswitch regulator, where the photoswitch regulator binds to the polypeptide, thereby generating a light-regulated polypeptide; and b) changing the wavelength of light to which the light-regulated polypeptide is exposed. In some embodiments, the present invention modulates the activity of a ligand-binding polypeptide, where the method generally involves: a) contacting the ligand-binding polypeptide with a photoswitch regulator, thereby generating a light-regulated polypeptide; and b) changing the wavelength of light to which the light-regulated polypeptide is exposed.

Changing in the wavelength of light to which the light-regulated polypeptide is exposed includes: 1) a change from λ₁ to λ₂; 2) a change from λ₂ to λ₁; 3) a change from λ₁ to darkness (no light); and 4) a change from darkness to λ₁. In certain aspects, the embodiments provides methods of modulating activity of a native (wild-type) polypeptide, where the method generally involves: a) contacting a polypeptide with a photoswitch regulator, where the photoswitch regulator binds to the polypeptide, forming a synthetic regulator/polypeptide complex; and b) changing the wavelength of light to which the synthetic regulator/polypeptide complex is exposed. A change in the wavelength of light to which the light-regulated polypeptide is exposed includes: 1) a change from λ₁ to λ₂; 2) a change from λ₂ to λ₁; 3) a change from λ₁ to darkness (no light); and 4) a change from darkness to λ₁. The photoswitch regulator/polypeptide complex is also referred to as a “light-regulated polypeptide.”

In some embodiments, the receptor or the light-regulated polypeptide is present in a cell-free in vitro system, e.g, the receptor or the light-regulated polypeptide is not associated with a cell. In other embodiments, the receptor or the light-regulated polypeptide is associated with a cell, e.g., the receptor or the light-regulated polypeptide is integrated into a cell membrane in a cell, the receptor or the light-regulated polypeptide is in the cytosol of a cell, the receptor or the light-regulated polypeptide is in an intracellular organelle, etc. In other embodiments, the receptor or the light-regulated polypeptide is in a synthetic membrane, e.g., in a planar synthetic membrane, in a liposome, in a membrane of an artificial cell, etc. In some embodiments, the cell-associated polypeptide or the cell-associated light-regulated polypeptide is in a cell in vitro, e.g., in a cell in a monolayer, in a cell in suspension, in an in vitro tissue, etc. In other embodiments, the cell-associated polypeptide or the cell-associated light-regulated polypeptide is in a cell in vivo, e.g., in a cell of an organism, e.g., a living organism.

In some embodiments, the change in wavelength (from λ₁ to λ₂; from light to darkness; or from darkness to light) results in a change in binding of the ligand to a ligand-binding site. A change in binding of a ligand to a ligand-binding site can include increased binding and decreased binding. Increased binding includes one or more of: an increased probability of binding of the ligand to the ligand-binding site; an increased binding affinity of the ligand for the ligand-binding site; an increased local concentration of the ligand at the ligand-binding site; and an increased occupancy of the ligand in the ligand-binding site. Decreased binding includes one or more of: a decreased probability of binding of the ligand to the ligand-binding site; a decreased binding affinity of the ligand for the ligand-binding site; a decreased local concentration of the ligand at the ligand-binding site; and a decreased occupancy of the ligand in the ligand-binding site. A change in wavelength to which a synthetic regulator is exposed, or to which a polypeptide/synthetic light regulator complex is exposed, can refer to a change in wavelength from λ₁ to λ₂; a change from light to darkness; or a change from darkness to light. An increase in binding includes an increase of from about 10% to about 50%, from about 50% to about 2-fold, from about 2-fold to about 5-fold, from about 5-fold to about 10-fold, from about 10-fold to about 50-fold, from about 50-fold to about 10²-fold, from about 10²-fold to about 10⁴-fold, from about 10⁴-fold to about 10⁶-fold, from about 10⁶-fold to about 10⁸-fold, or a greater than 10⁸-fold increase in binding. A decrease in binding includes a decrease of from about 5% to about 10% to about 20% to about 30%, from about 30% to about 40%, from about 40% to about 50%, from about 50% to about 60%, from about 60% to about 70%, from about 70% to about 80%, from about 80% to about 90%, or from about 90% to 100% decrease in binding.

For example, in some embodiments, the ligand has a first probability of binding to the ligand site at a first wavelength of light; the ligand has a second probability of binding to the ligand binding site at a second wavelength of light; and the second probability is lower than the first probability. In other embodiments, the ligand has a first probability of binding to the ligand site at a first wavelength of light; the ligand has a second probability of binding to the ligand binding site at a second wavelength of light; and the second probability is higher than the first probability. In other embodiments, ligand has a first probability of binding to the ligand site when exposed to light; the ligand has a second probability of binding to the ligand binding site in the absence of light (e.g., in darkness); and the second probability is lower than the first probability. In other embodiments, the ligand has a first probability of binding to the ligand site when exposed to light; the ligand has a second probability of binding to the ligand binding site in the absence of light and the second probability is higher than the first probability.

A change in wavelength can result in a change in activity of the light-regulated protein. The activity will depend, in part, on the polypeptide, and can include activity of an ion channel; activity of a receptor in transmitting a signal; etc.

In some embodiments, the change in wavelength results in binding of the ligand to the ligand-binding site of the polypeptide. In some embodiments, the change in wavelength results in increased binding affinity of the ligand to the ligand-binding site for the polypeptide. In these embodiments, where the ligand is an agonist, and the change results in activation of the polypeptide; and where the ligand is an antagonist, the change results in block of activation of the polypeptide; and where the ligand is an active site or pore blocker, the change results in inhibition of the polypeptide; and where the ligand is a blocker of a site of interaction with other macromolecules, the change interferes with that interaction. In some embodiments, prolonged binding of an agonist to the ligand-binding site results in desensitization or inactivation of the polypeptide. In other embodiments, binding of an antagonist blocks activation of the receptor.

In other embodiments, the change in wavelength results in lack of binding of the ligand to the ligand-binding site, e.g., removal of the ligand from the ligand-binding site of the polypeptide. In other embodiments, the change in wavelength results in reduced binding affinity of the ligand for the ligand-binding site, e.g., reduced binding affinity of ligand for the ligand-binding site of the polypeptide. In these embodiments, where the ligand is an antagonist, the change results in activation of said polypeptide; and where the ligand is an agonist, the change results in deactivation of light-regulated polypeptide, or recovery from desensitization or inactivation.

In some embodiments, the polypeptide/synthetic regulator complex is exposed to light of a first wavelength, where exposure to light of the first wavelength (λ₁) results in binding of the ligand to the ligand-binding site (or increased binding affinity of the ligand for the ligand-binding site); and the polypeptide/synthetic regulator complex is subsequently exposed to light of a second wavelength (λ₂), where exposure to light of the second wavelength results in removal of the ligand from the ligand-binding site (or reduced binding affinity of the ligand for the ligand-binding site). This change in wavelength from a first wavelength to a second wavelength (Δλ) can be repeated numerous times, such that the light is switched back and forth between λ₁ and λ₂. Switching between λ₁ and λ₂ results in switching or transition from a ligand-bound state to a ligand-unbound state.

In some embodiments, the polypeptide/synthetic regulator complex is exposed to light of a first wavelength, where exposure to light of the first wavelength (λ₁) results in binding of the ligand to the ligand-binding site (or increased binding affinity of the ligand for the ligand-binding site); and the light is subsequently turned off, e.g., the polypeptide/synthetic regulator complex is in darkness, where keeping the polypeptide/synthetic regulator complex in darkness results in removal of the ligand from the ligand-binding site (or reduced binding affinity of the ligand for the ligand-binding site). This change from λ₁ to darkness can be reversed, e.g., from darkness to λ₁; and repeated any number of times, as described above. In other embodiments, the polypeptide/synthetic regulator complex is exposed to light of a first wavelength, where exposure to light of the first wavelength (λ₁) results in lack of binding of the ligand to the ligand-binding site (or reduced binding affinity of the ligand for the ligand-binding site); and the light is subsequently turned off, e.g., the polypeptide/synthetic regulator complex is in darkness, where keeping the polypeptide/synthetic regulator complex in darkness results in binding of the ligand to the ligand-binding site (or increased binding affinity of the ligand for the ligand-binding site). This change from λ₁ to darkness can be reversed, e.g., from darkness to λ₁; and repeated any number of times, as described above.

As noted above, the change in wavelength can be repeated any number of times, e.g, from λ₁ to λ₂ and from λ₂ to λ₁; or from λ₁ to darkness and from darkness to λ₁. Thus, a method provides for inducing a transition or switch from a ligand-bound state of a protein to a ligand-unbound state of the protein, or from a high affinity state to a low affinity state. Depending on whether the ligand is an agonist or an antagonist, the light-regulated polypeptide will in some embodiments be switched from an active state to an inactive (or deactivated) state, or from an inactive (or deactivated) state to an active state.

The wavelength of light to which the light-regulated polypeptide is exposed ranges from 10⁸ m to about 1 m, e.g., from about 10⁻⁸ m to about 10⁻⁷ m, from about 10⁻⁷ m to about 10⁻⁶ m, from about 10⁻⁶ m to about 10⁻⁴ m, from about 10⁻⁴ m to about 10⁻² m, or from about 10⁻² m to about 1 m. “Light,” as used herein, refers to electromagnetic radiation, including, but not limited to, ultraviolet light, visible light, infrared, and microwave.

The wavelength of light to which the light-regulated polypeptide is exposed ranges in some embodiments from about 200 nm to about 800 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, or from about 750 nm to about 800 nm, or greater than 800 nm.

In other embodiments, the wavelength of light to which the light-regulated polypeptide is exposed ranges from about 800 nm to about 2500 nm, e.g., from about 800 nm to about 900 nm, from about 900 nm to about 1000 nm, from about 1000 nm to about 1200 nm, from about 1200 nm to about 1400 nm, from about 1400 nm to about 1600 nm, from about 1600 nm to about 1800 nm, from about 1800 nm to about 2000 nm, from about 2000 nm to about 2250 nm, or from about 2250 nm to about 2500 nm. In other embodiments, the wavelength of light to which the light-regulated polypeptide is exposed ranges from about 2 nm to about 200 nm, e.g., from about 2 nm to about 5 nm, from about 5 nm to about 10 nm, from about 10 nm to about 25 nm, from about 25 nm to about 50 nm, from about 50 nm to about 75 nm, from about 100 nm, from about 100 nm to about 150 nm, or from about 150 nm to about 200 nm.

The difference between the first wavelength and the second wavelength can range from about 10 nm to about 800 nm or more, e.g., from about 10 nm to about 25 nm, from about 25 nm to about 50 nm, from about 50 nm to about 100 nm, from about 100 nm to about 200 nm, from about 200 nm to about 250 nm, from about 250 nm to about 500 nm, or from about 500 nm to about 800 nm. Of course, where the light-regulated polypeptide is switched from darkness to light, the difference in wavelength is from essentially zero to a second wavelength.

The intensity of the light can vary from about 1 W/m² to about 50 W/m², e.g., from about 1 W/m² to about 5 W/m², from about 5 W/m² to about 10 W/m², from about 10 W/m², from about 10 W/m² to about 15 W/m², from about 15 W/m² to about 20 W/m², from about 20 W/m² to about 30 W/m², from about 30 W/m² to about 40 W/m², or from about 40 W/m² to about 50 W/m². The intensity of the light can vary from about 1 μW/cm² to about 100 μW/cm², e.g., from about 1 μW/cm² to about 5 μW/cm², from about 5 μW/cm² to about 10 μW/cm², from about 10 μW/cm² to about 20 μW/cm², from about 20 μW/cm² to about 25 μW/cm², from about 25 μW/cm² to about 50 μW/cm², from about 50 μW/cm² to about 75 μW/cm², or from about 75 μW/cm² to about 100 μW/cm². In some embodiments, the intensity of light varies from about 1 μW/mm² to about 1 W/mm², e.g., from about 1 μW/mm² to about 50 μW/mm², from about 50 μW/mm² to about 100 μW/mm², from about 100 μW/mm² to about 500 μW/mm², from about 500 μW/mm² to about 1 mW/mm², from about 1 mW/mm² to about 250 mW/mm², from about 250 mW/mm² to about 500 mW/mm², or from about 500 mW/mm² to about 1 W/mm².

In some embodiments, the light-regulated polypeptide is regulated using sound, instead of electromagnetic (EM) radiation (light). For example, in some embodiments, the light-regulated polypeptide is regulated using ultrasound to effect a change from a first isomeric form to a second isomeric form.

The duration of exposure of the light-regulated polypeptide to light can vary from about 1 μsecond (μs) to about 60 seconds (s) or more, e.g., from about 1 μs to about 5 μs, from about 5 μs to about 10 μs, from about 10 μs to about 25 μs, from about 25 μs to about 50 μs, from about 50 μs to about 100 μs, from about 100 μs to about 250 μs, from about 250 μs to about 500 μs, from about 500 μs to about 1 millisecond (ms), from about 1 ms to about 10 ms, from about 10 ms to about 50 ms, from about 50 ms to about 100 ms, from about 100 ms to about 500 ms, from about 500 ms to about 1 second, from about 1 second to about 5 seconds, from about 5 seconds to about 10 seconds, from about 10 seconds to about 15 seconds, from about 15 seconds to about 30 seconds, from about 30 seconds to about 45 seconds, or from about 45 seconds to about 60 seconds, or more than 60 seconds. In some embodiments, the duration of exposure of the light-regulated polypeptide to light varies from about 60 seconds to about 10 hours, e.g., from about 60 seconds to about 15 minutes, from about 15 minutes to about 30 minutes, from about 30 minutes to about 60 minutes, from about 60 minutes to about 1 hour, from about 1 hour to about 4 hours, from about 4 hours to about 6 hours, from about 6 hours to about 8 hours, or from about 8 hours to about 10 hours, or longer.

The duration of binding of the ligand portion of the synthetic regulator to the ligand-binding site can vary from less than one second to days. For example, in some embodiments, the duration of binding of the ligand portion of the synthetic regulator to the ligand-binding site varies from about 0.5 second to about 1 second, from about 1 second to about 5 seconds, from about 5 seconds to about 15 seconds, from about 15 seconds to about 30 seconds, from about 30 seconds to about 60 seconds, from about 1 minute to about 5 minutes, from about 5 minutes to about 15 minutes, from about 15 minutes to about 30 minutes, or from about 30 minutes to about 60 minutes. In other embodiments, the duration of binding of the ligand portion of the synthetic regulator to the ligand-binding site varies from about 60 minutes to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, from about 36 hours to about 48 hours, from about 48 hours to about 60 hours, from about 60 hours to about 72 hours, from about 3 days to about 4 days, from about 4 days to about 5 days, or from about 5 days to about 7 days, or longer.

(g) Additional Proteins

In some embodiments, the cells further express one or more proteins that extend the range of the membrane potential change that results from the activity of the photoswitchable channel. In some embodiments, these proteins are exogenous to the cells. In some embodiments, these proteins are native. In some embodiments, the proteins form pores or channels in the cell membrane, and can allow various compounds, e.g., ions, to enter or exit the cell. In some embodiments, the one or more proteins are permeable to ions, e.g., potassium ions, and facilitate hyperpolarization of the cell.

In some embodiments, the one or more proteins that extend the range of the membrane potential change comprise inward rectifier channels. Inward rectifier channels pass current, e.g., positive charge, more easily in the inward direction, i.e., into the cell. In some embodiments, these channels are used in the assays of the present invention to facilitate loading of charge, e.g., positively charged K+ ions, into the cell. In some embodiments, the inward rectifier channel comprises Kir2.1. In some embodiments, the inward rectifier channels comprise G protein-coupled inwardly-rectifying potassium channels (GIRKs). In some embodiments, the one or more proteins comprise one or more of Kir1.1, Kir2.1, Kir2.2, Kir2.3, Kir2.4, Kir3.1 (GIRK1), Kir3.2 (GIRK2), Kir3.3 (GIRK3), Kir3.4 (GIRK4), Kir4.1, Kir4.2, Kir5.1, Kir6.1, Kir6.2 and Kir7.1.

In some embodiments, the one or more proteins that extend the range of the membrane potential change comprise active transporters. Primary active transporters use energy, e.g., in the form of adenosine triphosphate (ATP), to drive compounds across the cell membrane. P-type ATPases are primary active transporters that can transport cations across cell membranes, e.g., Na+, K+, Ca2+ or H+. Primary active transporters P-type ATPase that can be used to extend the range of membrane potential include: 1) P-type ATPases, e.g., sodium potassium pumps, calcium pump and proton pumps; 2) F-ATPases, e.g., mitochondrial ATP synthase and chloroplast ATP synthase; 3) V-ATPases, e.g., vacuolar ATPase; and 4) ABC (ATP Binding Cassette) transporters, e.g., MDR and CFTR. In some embodiments, the active transporters used in the present invention comprise electrogenic pumps. In some embodiments, the electrogenic pumps comprise sodium-potassium pumps, e.g., the sodium-potassium pump Na⁺/K⁺ ATPase. Sodium-potassium pumps actively transport sodium and potassium across the cell membrane. For example, the Na+/K+ ATPase pump can transport 3 Na+ ions out of the cell in exchange for transporting 2 K+ ions into the cell. The activity of the N+-K+-ATPase can be modified by exogenous compounds. For example, cardiac glycosides, e.g., digoxin and ouabain, can downregulate its activity. Alternately, the Na+/K+-ATPase is upregulated by cyclic adenosine monophosphate (cAMP). Secondary active transporters are not directly coupled to ATP. These transporters use an electrochemical potential difference created by pumping ions out of the cell to drive another ion across the membrane. Counter-transporters pump two compounds across the membrane in opposite directions. For example, the sodium-calcium exchanger, or antiporter, allows three sodium ions into the cell to transport one calcium ion out of the cell. Co-transporters pump two compounds in the same direction across a membrane but in opposite concentration gradients. For example, the glucose symporter SGLT1 co-transports one glucose or galactose molecule into a cell for every two sodium ions it transports into the cell. In some embodiments, secondary active transporters are used to enhance the membrane potential change resulting from modulation of the photoswitchable ion channel.

(h) Ion Channel Activity Readouts

There are a variety of means for optically determining ion channel activity in a cell. In some embodiments, ion channel activity and/or the effect of a compound on a test channel is determined by determining the membrane potential of a cell. In some embodiments, ion channel activity and/or the effect of a compound on a test channel is determined by determining a change in ion concentration (e.g. intracellular or extracellular concentration). In the present invention, the photoswitchable ion channel provides a means to alter membrane potential, and ion concentrations, often in a very rapid manner. The readout should have comparable response time. In some embodiments, determining the membrane potential of the cells comprises one or more of an optical measurement and an electrical measurement. In some embodiments, determining a change in the concentration of an ion comprises an optical measurement. In some embodiments, the optical measurement comprises detecting a voltage-sensitive dye fluorescence, an ion-sensitive dye fluorescence, a voltage sensitive fluorescence resonance energy transfer (FRET), or a nanocrystal luminescence. Suitable methods for measuring the membrane potential include the following:

(i) Fast Voltage-Sensitive Dyes (VSD)

Fast VSDs undergo charge redistribution in the excited state, relative to the ground state, resulting in a large change in the dipole moment. The transition energy between the two states is accordingly dependent on the value of the local electric field. As this mechanism is electronic and does not require translocation or reorientation of the molecule, the response time can be much faster than the ms-time scale changes in the membrane potential with some photoswitched ion channel currents.

In some embodiments, the excitation wavelength range for the voltage-sensitive dye does not overlap with the wavelength ranges for controlling the photoswitched ion channel. In some embodiments, the invention makes use of red-shifted VSDs, several of which have been reported in recent years. See, e.g., Wuskell, J P et al., Synthesis, spectra, delivery and potentiometric responses of new styryl dyes with extended spectral ranges, (2006) Journal of Neuroscience Methods 151 200-215; G. Salama, et al., Properties of New, Long-Wavelength, Voltage-sensitive Dyes in the Heart. (2006) J. Membrane Biol. 208, 125-140. These dyes are at least as sensitive as the 1-(3-sulfonatopropyl 4-[β[2-(di-n-octylamino)-6-naphthyl]vinyl]pyridinium betaine (ANEPPs) dyes that have been the standard for fast VSDs. Other red-shifted VSDs have been reported. See, e.g., Kuhn, B and Fromherz, P, Anellated Hemicyanine Dyes in a Neuron Membrane: Molecular Stark Effect and Optical Voltage Recording, (2003) J. Phys. Chem. B 107, 7903-7913. For dyes with low fluorescence quantum efficiency in aqueous solution, e.g., as is the case with the styryl dyes, then only dye bound to the membrane contributes significantly to the fluorescence. Thus, the staining level should not affect the voltage-dependent relative fluorescence change, ΔF/F, because both the numerator and denominator of ΔF/F change proportionally to the degree of staining.

(ii) Fluorescence Resonance Energy Transfer (FRET)

In some embodiments, FRET is used to measure the membrane potential by monitoring the change in fluorescence of the donor or the acceptor due to a change in the separation of the two chromophores constituting the FRET pair. One of the pair is membrane immobilized, and the other moves between the inner and outer leaflets of the membrane as the voltage gradient across the membranes changes. In one embodiment, the FRET process is between a genetically encoded probe, membrane-anchored enhanced Green Fluorescent Protein (eGFP) (the donor), and dipicrylamine, a non-fluorescent, hydrophobic anion that partitions into the plasma membrane (the acceptor). The movement of the dipicrylamine in response to changes in membrane potential produces a 34% change in the fluorescence of the eGFP per 100 mV, with a response time of 0.5 ms. The dipicrylamine moves from the outer to the inner leaflet on depolarization: As the inner leaflet becomes less negative, the anion redistributes.

A number of FRET pairs can be used with the present invention. Generally, the principals for choosing the mobile species include the following: 1) the hydrophobic species is anionic, as biological membranes contain ester groups that create dipole potential within the membrane that cause anions to have greater mobility than cations within the membrane (Flewellling and Hubbell 1986); 2) Increasing hydrophobicity increases the fraction of dye resident in the plasma membrane, e.g., in the interior of the membrane. Being buried in the membrane decreases the activation energy for translocation from one leaflet to the other. Conversely, the existence of a polar group can increase the solvation energy in the headgroup region of the membrane, increasing the activation energy for translocation; 3) Delocalization of the ionic charge reduces the Born charging energy, in proportion to 1/r, r being the radius of the anion, required to move a charged species from a hydrophilic to a hydrophobic environment (Benz 1988).

In some embodiments, the voltage sensor is a hydrophobic fluorescent anion that binds tightly to the plasma membrane at two energy minima at the intracellular and extracellular membrane-water interfaces. The impermeant fluorophore is located on the extracellular face of the plasma membrane and functions as the donor, while the hydrophobic fluorescent anion, an oxonol moiety, functions as the acceptor. At normal resting membrane potentials, the oxonol molecules bind predominantly to the extracellular membrane surface resulting in FRET. Upon depolarization, the oxonol molecules translocate to the inner leaflet and FRET is diminished, causing an enhancement of the donor emission and a decrease in the acceptor emission.

(iii) Nanocrystals

In some embodiments, the present invention monitors membrane potential using nanocrystal luminescence. The Stark effect, the shift of the absorption as a function of electric field, is the physical phenomenon underlying the voltage sensitivity of the fast VSDs and has been reported in nanocrystals, e.g., in semiconductor nanocrystals. The electric field across the plasma membrane is relatively large, ˜50 mV/5 nm=10⁵ V/cm. In addition to shifting the transition energy of the exciton band, the local electric field affects the excited state lifetime. In an electric field, the probability that an excited carrier will be trapped by a surface state of the nanocrystal is increased. In some embodiments, the present invention makes use of modified nanoparticles (phospholipid-coated EviTag-T2 (Evident Technologies, Troy, N.Y.)). See PCT Patent Application, WO/2006/096835, “Monitoring and Manipulating Cellular Transmembrane Potentials Using Nanostructures” to Molokanova et al. 2008. In some embodiments, mono-disperse CdSe and CdSe/CdS core/shell nanocrystals are used. A 7-fold increase in fluorescence intensity in response to membrane depolarization induced had been observed with such nanocrystals by addition of KCl. The change is membrane potential was estimated to be 54 mV (www.sandia.gov/mission/step/stories/2008/August/FanFinal.pdf). These nanocrystals can be synthesized by a so-called hot soap injection process. Phospholipids can be used to encapsulate the nanocrystals within a micelle core, forming water-soluble, biocompatible nanoparticles. The interdigitated surfactant layers surrounding the nanoparticles enable fusion into the cell membrane.

(iv) Ion-Sensitive Dyes

Ion-sensitive dyes include those dyes that exhibit changes in fluorescence when bound to an ion. Ion-sensitive dyes can be used to detect cations or anions, including but not limited to, sodium ions, potassium ions, calcium ions, chloride ions, sulphate ions, magnesium ions, and other metal ions. Ion-sensitive dyes can be used to detect levels of and/or changes in intracellular or extracellular ion concentrations. Dyes can be used alone or in combination with other dyes or detection agents. Examples of ion-sensitive dyes include, but are not limited to, Fura-2, Fluo-3, Rhod-2, Fura-C18, Fura-FF-C18, Calcium Green C18, Mag-indo-1, Mag-fura-2, FluoZin-1, FuraZin-1, Phen Green FL, NewportGreen, Nitrophyl EGTA, DMNP-EDTA, Diazo-2, BAPTA, TPEN, SNARF-1, SBFI, PBFI, SPQ, MQAE, MEQ, lucigenin L-6868, and their AM-esters. In some embodiments, the excitation wavelength range for the voltage-sensitive dye does not overlap with the wavelength ranges for controlling the photoswitched ion channel.

In some embodiments, one or more ion-sensitive dyes are used to detect changes in calcium ion (Ca²⁺) concentration. Fluorescent probes having a spectral response upon binding Ca²⁺ can be used to observe changes in the intracellular free Ca²⁺ concentration. Examples of these fluorescent indicators include, but are not limited to, derivatives of Ca²⁺ chelators, such as EGTA (ethylene glycol tetraacetic acid) and BAPTA (1,2-bis(o-aminophenoxy)ethane-N,N,N′,N′-tetraacetic acid). Fluorescent indicators optimized for detecting changes in intracellular Ca²⁺ over the range of <50 nM to >50 μM are offered by Invitrogen (Molecular Probes). These include the following: fura-2, indo-1, fluo-3, fluo-4, rhod-2, Oregon Green 488 BAPTA, Calcium Green, X-rhod-1 and Fura Red; fura-4F, fura-6F and fura-FF provide increased sensitivity to intracellular Ca²⁺ concentration in the 0.5-5 μM range. Indicator dyes, such as ion-sensitive dyes, can be conjugated to high- or low-molecular weight dextrans for improved cellular retention. In a one embodiment, the dye is a cell-permeant acetoxymethyl (AM) ester, which can be passively loaded into cells where it is cleaved to cell-impermeant products by intracellular esterases.

One example of a calcium ion-sensitive dye, Fluo-3, is essentially nonfluorescent unless bound to Ca²⁺ and exhibits a quantum yield at saturating Ca²⁺ of ˜0.14 and a Kd for Ca²⁺ of 390 nM. The intact acetoxymethyl (AM) ester derivative of fluo-3 is almost nonfluorescent, unlike the AM esters of fura-2 and indo-1. The fluorescent emission of Ca²⁺-bound fluo-3 is green (˜525 nm). Another example of a calcium ion-sensitive dye, Fluo-4, an analog of fluo-3 with the two chlorine substituents replaced by fluorine atoms, exhibits a Kd for Ca²⁺ of 345 nM and an excitation wavelength optimized for 488-nm excitation.

In some embodiments, an ion-sensitive indicator for measuring changes in ion concentration is a protein. In some embodiments, an ion-sensitive indicator is a protein-based Ca²⁺ sensor. One class of indicator is composed of a Ca²⁺-responsive element, such as calmodulin, inserted into a fluorescent protein, such that Ca²⁺ binding alters the spectral properties of the chromophore. Examples of this class include, but are not limited to, the camgaroos G-CaMPs, pericams, “Case” sensors, and grafted EF-hands. An additional Ca²⁺ sensor class is the “cameleon-type” which have Ca²⁺-responsive elements inserted between two fluorescent proteins, such that an alteration in the efficiency of fluorescence resonance energy transfer (FRET) occurs between the two FPs when the responsive element binds Ca²⁺.

In some embodiments, one or more ion-sensitive dyes are used to detect changes in potassium ion (K⁺) concentration. Measuring ion flux through potassium channels in a cell-based assay has been described (Weaver et al., 2004, Journal of Biomolecular Screening 9 671-677; incorporated herein by reference in its entirety), based on the permeability of Tl⁺ through K⁺ channels and the use of a Tl⁺-sensitive fluorescent dye.

The AM ester of the Tl⁺-sensitive dye, BTC-AM (Invitrogen), can be loaded into cultured cells by removing the medium and replacing it with a solution of dye in a Cl⁻-free buffer, after which the loading solution can be removed and replaced with the Cl⁻-free assay buffer. Tl⁺ can be added to the extra-cellular medium in a solution of Tl₂SO₄ to a final concentration in the range of approximately 1-10 mM. Alternatively, use of the reagent formulation FluxOR™ (Invitrogen), allows the use of the dye in physiological saline, without the need to load or assay cells in chloride-free conditions. Thallium chloride is an insoluble precipitate that forms when concentrations of free thallium and chloride in the solution are greater than about 4 mM. FluxOR™ can be used at a final concentration of 2 mM.

(v) Electrical

In some embodiments, the present invention uses electrical, rather than optical, detection technology. Compared to optical measure, electrical readout does not require voltage-sensitive fluorescent entities, the corresponding light source, the attendant optics or a detector. Instead, an electrode, or array of electrodes, can be used in each well. In some embodiments, a corresponding set of amplifiers is used.

In some embodiments, the present invention uses systems that measure the impedance of a layer of cells. The electrical readout embodiment of this invention has an array of electrodes on the bottom surface of the sample well, each electrode being of a diameter that is approximately that of a typical mammalian cell, i.e., ˜10-20 μm. The readout can be performed on a well-by-well basis, performing a sum over the induced potentials of all the electrodes in the well.

IV. Instrumentation

(a) Properties of the Excitation Sources

Properties of the excitation source can be used to modulate photoswitchable ion channels. In some embodiments, the change in the membrane potential induced by the light is a function of the intensity. In some embodiments, the value of the membrane potential is set by adjusting the intensity of the source that activates the photoswitched channel. In some embodiments, the value of the membrane potential is set by adjusting the intensity of the excitation source that inactivates the photoswitched channel. In some embodiments, both adjustments are used, e.g., to repeatedly activate and deactivate the photoswitchable ion channel.

In some embodiments, e.g., using currently available azobenzene photoswitches, a source intensity of approximately 5 mW/mm² provides for fast, e.g., 1 ms, changes in membrane potential, consequent to a change in photoswitched ion channel conductance. Depending on the photoswitch, the source intensity and other factors, the time to change the membrane potential can be set over a large range, e.g., from 10⁻¹⁴ s to 100 s. In some embodiments, the source intensity is modulated to cause a change in the membrane potential is about 10⁻¹⁴ s, 10⁻¹³ s, 10⁻¹² s, 10⁻¹¹ s, 10⁻¹⁰ s, 10⁻⁹ s, 10⁻⁸ s, 10⁻⁷ s, 10⁻⁶ s, 10⁻⁵ s, 10⁻⁴ s, 10⁻³ s, 0.01 ms, 0.05 ms, 0.10 ms, 0.5 ms, 1 ms, 2 ms, 3 ms, 4 ms, 5 ms, 6 ms, 7 ms, 8 ms, 9 ms, 10 ms, 15 ms, 20 ms, 25 ms, 30 ms, 40 ms, 50 ms, 60 ms, 70 ms, 80 ms, 90 ms, 100 ms, 150 ms, 200 ms, 250 ms, 300 ms, 400 ms, 500 ms, 600 ms, 700 ms, 800 ms, 900 ms, 1 s, 2 s, 3 s, 4 s, 5 s, 6 s, 7 s, 8 s, 9 s, 10 s, 15 s, 20 s, 25 s, 30 s, 40 s, 50 s, 60 s, 70 s, 80 s, 90 s, or 100 s. In some embodiments, the source intensity is modulated to cause a change in the membrane potential in more than 100 s. Consider an example, provided for illustrative purposes only, wherein the assays are performed in a 384-well microtiter plate, wherein all wells are illuminated simultaneously and wherein approximately 5 mW is delivered to each well over an illumination area of about 1 mm². Under these conditions, the source power requirement is approximately (5×10⁻³)*(384) or 2 W. At these intensities, a few percent of the photoswitches will be flipped per millisecond of illumination, and the response will be linear over a wide range of intensity and/or duration of excitation illumination.

In some embodiments, solid-state illumination sources are used, e.g., semiconductor diode-based, either light-emitting diodes (LEDs) or lasers. In some embodiments, semiconductor diode pumped solid state (DPSS) lasers are used. LEDs have the advantage of lower cost. Lasers have the advantage of superior spatial beam profiles, making it easier to efficiently use the emitted radiation, as the beams are generally better collimated and the effective source size is smaller. The present invention provides optical configurations using both types of sources, as described below.

In some embodiments, the kinetic curve of the assay is approximately 10-20 ms in duration; the photoswitched channel can be reset in approximately 10 ms. Depending on the photoswitch, the source intensity and other factors, the time to reset the photoswitched channel can be set over a large range, e.g., from 10⁻¹⁴ s to 100 s. In some embodiments, the photoswitched channel can be reset in about 10⁻¹⁴ s, 10⁻¹³ s, 10⁻¹² s, 10⁻¹¹ s, 10⁻¹⁰ s, 10⁻⁹ s, 10⁻⁸ s, 10⁻⁷ s, 10⁻⁶ s, 10⁻⁵ s, 10⁻⁴ s, 10⁻³ s, 0.01 ms, 0.05 ms, 0.10 ms, 0.5 ms, 1 ms, 2 ms, 3 ms, 4 ms, 5 ms, 6 ms, 7 ms, 8 ms, 9 ms, 10 ms, 15 ms, 20 ms, 25 ms, 30 ms, 40 ms, 50 ms, 60 ms, 70 ms, 80 ms, 90 ms, 100 ms, 150 ms, 200 ms, 250 ms, 300 ms, 400 ms, 500 ms, 600 ms, 700 ms, 800 ms, 900 ms, 1 s, 2 s, 3 s, 4 s, 5 s, 6 s, 7 s, 8 s, 9 s, 10 s, 15 s, 20 s, 25 s, 30 s, 40 s, 50 s, 60 s, 70 s, 80 s, 90 s, or 100 s. In some embodiments, the experiment is repeated at 0.001-1000 Hz. In some embodiments, the experiment is repeated at 1-100 Hz. In some embodiments, the experiment is repeated at 1-50 Hz. In some embodiments, the experiment is repeated at 10-50 Hz. In some embodiments, the experiment is repeated at 10-30 Hz. In some embodiments, the experiment is repeated at about 0.001 Hz, 0.005 Hz, 0.01 Hz, 0.05 Hz, 0.1 Hz, 0.5 Hz, 1 Hz, 5 Hz, 10 Hz, 15 Hz, 20 hz, 25 hz, 30 Hz, 35 Hz, 40 Hz, 50 Hz, 60 Hz, 70 Hz, 80 Hz, 90 Hz, 100 Hz, 200 Hz, 300 Hz, 400 Hz, 500 Hz, 600 Hz, 700 Hz, 800 Hz, 900 Hz, or about 1000 Hz. In a few seconds, many repeat scans can be averaged. In some embodiments, the signals are averaged over a number of replicates from the same sample. In some embodiments, 10-100 replicates are averaged. In some embodiments 2-1000 replicates are averaged. In some embodiments, about 2, 5, 10, 15, 20, 25, 30, 40, 50, 60, 70, 80, 90, 100, 150, 200, 250, 300, 400, 500, 600, 700, 800, 900, or about 1000 replicates are averaged. In some embodiments, more than 1000 replicates are averaged. The repeated measurements allow accurate identification of small effects. That a large number of replicates can be obtained in a few seconds allows a great deal of flexibility in optimizing the protocol for acquiring data from the entire plate.

Assay systems can be categorized by the number of runs that that can be performed per day. The assays of the present invention can be performed in a high throughput manner, e.g., at more than 100,000 assays/day. For illustrative purposes only, consider a daily throughput for an 8-hr day (28,800 s) for a system that measures, on average 2 wells/s. Such a system requires 3.2 minutes to read a 384-well plate, which corresponds to 57,000 assays/8 hrs, or more than 100,000 assays/day for two 8 hr shifts. Given the above estimates and accounting for dead time for changing plates, etc, a system that read ˜10 wells in parallel provides high-throughput capabilities. The present invention provides these capabilities.

(b) Voltage Sensitive Dye (VSD) Signal

The present invention provides systems for rapidly and accurately reading changes in membrane potential. These systems can take advantage of the fast read times provided by the rapidly changing the membrane potential of the photoswitch assays. Consider a non-limiting exemplary embodiment wherein the concentration of the readout fluorophore is approximately 1 μM. For a sample volume described by a 1 mm² area, 1 cell layer thick, or approximately 10⁻⁵ cm⁻³, there are approximately 6×10⁹ fluors. Further assuming the excitation source has an average power of 5 mW, the probability of absorption is approximately 15%/ms. And for a moderate emission quantum yield and a collection-detection efficiency of 0.1%, 3×10⁵ photons are detected for which the shot noise, a type of electronic noise that occurs when the finite number of particles that carry energy, such as electrons in an electronic circuit or photons in an optical device, is small enough to give rise to detectable statistical fluctuations in a measurement, is insignificant.

Further considering the exemplary embodiment, to achieve a collection-detection efficiency of 0.1%, the numerical aperture (NA) of the objective lens needs to be reasonably high. The fraction of the fluorescence that is collected by the lens is ½(1−cos(θ)), which for small θ is approximately sin²(θ)/4, or NA²/4, in air.

TABLE 4
Collection-detection efficiencies

| NA | Collection Fraction | Angle |
|---|---|---|
| 0.6 | 0.10 | 37 |
| 0.4 | 0.04 | 24 |
| 0.25 | 0.016 | 15 |
| 0.10 | 0.0025 | 06 |

(c) Optical Configurations

Any number of optical detection devices can be used with the methods and systems of the present invention. For example, known commercially available detection devices can be used. Various combinations of devices such as lenses and/or mirrors may be included. The optical devices may be used to redirect light, diffuse light, focus light, filter or block one or more wavelengths of light, alter one or more characteristics of emitted light, split light, and/or relay light. The following are non-limiting examples of optical detection devices and configurations contemplated by the present invention.

(i) Configuration 1

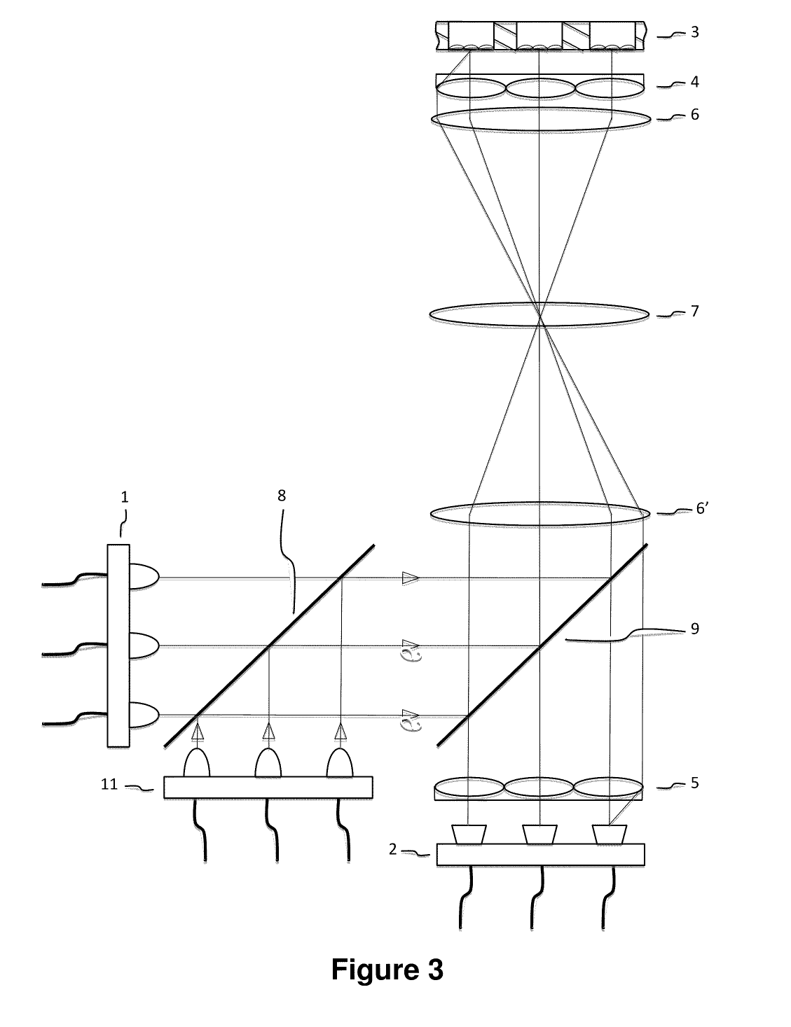

The present invention provides a variety of optical configurations. In one embodiment, a version of which is illustrated in FIG. 3, comprises an array of individual LEDs, arranged in the same pattern as the sample array, or a portion, thereof. The instrument can have multiple source arrays, e.g., two or three such arrays, exemplified in FIG. 3 as array 1 and array 11. Each source array can produce an array of collimated beams. One or more dichroic beam combiners 8 direct the excitation beams of different wavelengths to the sample array. A dichroic fluorescence beamsplitter 9 can separate the light, e.g., separating fluorescence from excitation radiation. A sample lens array 4 images the excitation beams onto, and collects the fluorescence from, the sample array 3. A detector lens array 5 images the fluorescence onto the detector array 2. In some embodiments, one or more of a telescope 6, 6′ and field lens 7 are used to relay the image of the sample array onto the detector array. In some embodiments, the source, sample and detector arrays are on the same pitch.

(ii) Configuration 1a

FIG. 4 illustrates a compact version of the instrument layout described above as shown in FIG. 3. As described above, the instrument retains array 1 and array 11, dichroic beam combiners 8, dichroic fluorescence beamsplitter 9, lens array 4, sample array 3, lens array 5, and detector array 2. But in this exemplary embodiment, the optional telescope 6, 6′ and the field lens 7 are omitted.

(iii) Configurations 1aTM

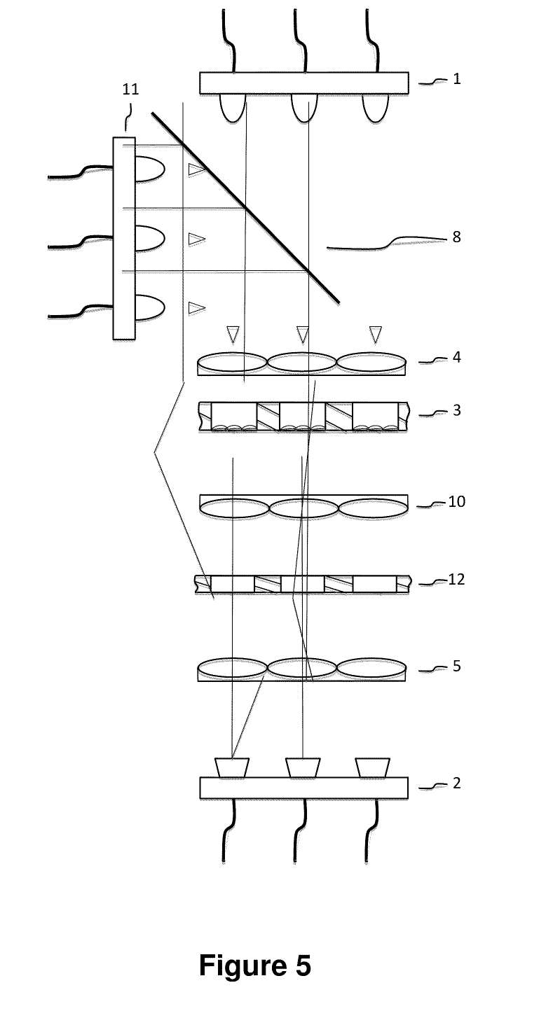

FIG. 5 illustrates an embodiment of a transmission geometry instrument. This configuration separates the illumination and detection apparatus on opposite sides of the sample array. As described above, the instrument retains source array 1, dichroic beam combiners 8, lens array 4, sample array 3, lens array 5, and detector array 2. In the exemplary arrangement shown in FIG. 5, the fluorescence collection lens array 10 and the corresponding detector lens array 5 are placed so that there is an intermediate image plane at the position of the spatial filter array 12. Spatial filtering in the intermediate image plane can reduce the background due to scattered light and fluorescence from adjacent samples of the sample array. It can also provide a degree of background rejection from fluorescence that originates from the solution above the plane of the sample cell layer. In addition to transmission illumination, these principles can be applied to the epifluorescence configurations depicted in FIG. 3 and FIG. 4, as well.

(iv) Configuration 2

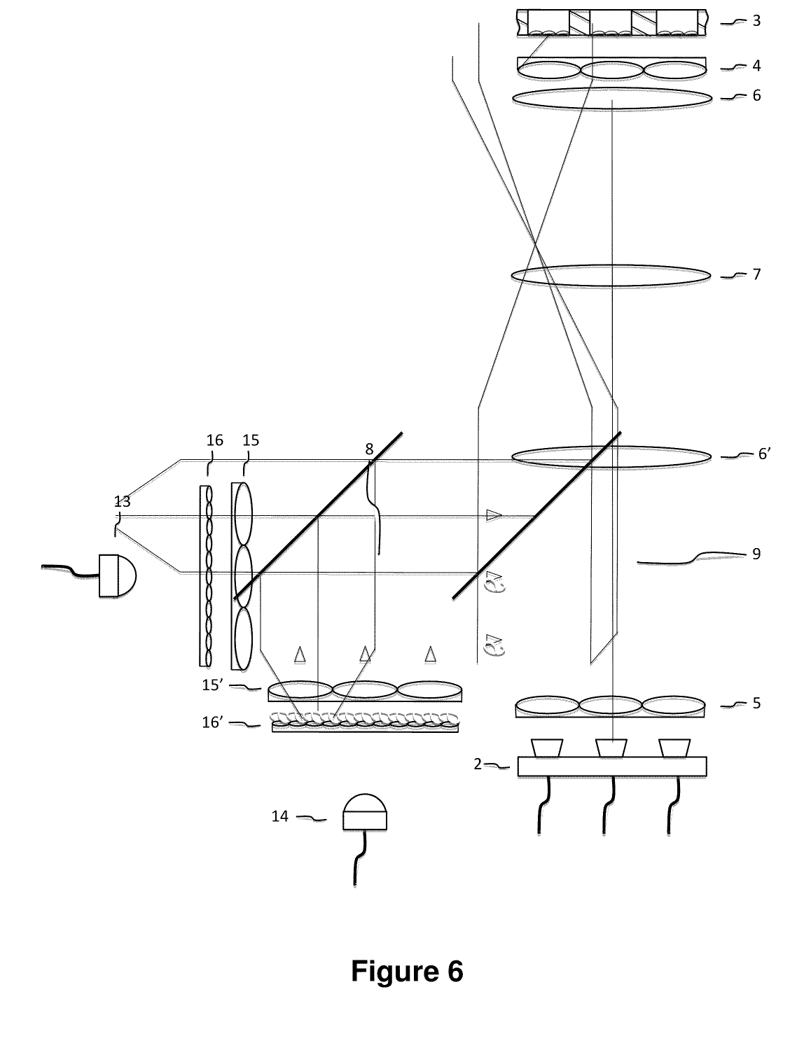

Another embodiment of an optical detection device, referred to as Configuration 2, is illustrated in FIG. 6. Configuration 2 comprises multiple, e.g., two, three or more, excitation sources 13 and 14, each of which can emit a different wavelength. Separate optics can be used for each source that include one of the following: a) conventional lenses, producing single beams, or b) microlens arrays 16 and 16′, producing flattop beams in circular or rectangular distributions, or producing rectangular arrays of individual beams. In some embodiments, lens arrays, 15, 15′, are used to collimate the illumination transmitted by the microlens arrays. The device further comprises one, two or more dichroic beam combiners 8 and dichroic fluorescence beamsplitter 9. A lens array 4 images the excitation beams onto, and collects the fluorescence from, the sample array 3. Another lens array 5 images the fluorescence onto the detector array 2. In some embodiments, one or more telescopes 6, 6′ and/or field lens 7 are used to magnify or demagnify the sample array onto the detector array. In some embodiments, the illumination is over the entire sample array, e.g., a 384-well microtiter plate. In some embodiments, the illumination is of a subset of the sample array, e.g., quadrants or quadrants of quadrants, rows, or other rectangular regions.

(v) Configuration 2T

The illumination sources and corresponding optical elements exemplified in Configurations 2 can also be arranged in a transmission geometry comprising two, three or more excitation sources, each emitting a different wavelength; separate optics for each source that include one of the following: a) conventional lenses, producing single beams, or b) microlens arrays, producing a flattop beams in circular or rectangular distributions, or producing rectangular arrays of individual beams; one (two) dichroic beam combiners; one embodiment of the collection-detection optics comprises a lens array to collect fluorescence from the sample array, a lens array to image the fluorescence onto the detector array, and optionally, a telescope and/or field lens to (de-)magnify the sample array onto said detector array. In some embodiments, the collection-detection optics comprises a lens array to collect fluorescence emission and form an image thereof in an intermediate image plane, a spatial filter array disposed in said intermediate image plane, and a detector lens array to relay the fluorescence image onto the detector array.

(vi) Sample Arrays

In some embodiments, the devices and systems of the present invention comprise a sample array. In some embodiments, the sample array comprises a multi-well plate. In some embodiments, the sample array comprises a microtiter plate. In some embodiments, the sample array comprises at least 6, 12, 24, 48, 72, 96, 384, 768, 1536, or more wells. Microtiter and multi-well plates suitable for use in the devices and systems of the invention include commercially available varieties known in the art, such as those sold by BioRad Laboratories (Hercules, Calif.), Life Technologies (Carlsbad, Calif.), Sigma-Aldrich (St. Louis, Mo.), Thermo Fisher Scientific (Rochester, N.Y.), and others. In some embodiments, the plate comprises wells comprising one or more transparent or translucent surfaces, such as a bottom surface, a wall, a top surface, or a combination thereof. In some embodiments, one or more surfaces of a well in a sample array comprise a material that selectively allows transmission of one or more wavelengths of light. In some embodiments, one or more surfaces of a well in a sample array comprise a material that selectively blocks transmission of one or more wavelengths of light.

In some embodiments, cells added to the sample array are allowed to adhere and/or grow prior to subjecting the sample to an assay. Cells may adhere to any surface or a selected surface of a well. Cells in the sample array can be subjected to an assay as adherent cells, cells in suspension, or a combination of these. In some embodiments, the sample array is sealed throughout an assay. In some embodiments, the contents of the wells are manipulated at one or more points during an assay, such as by the removal of well contents and/or addition of reagents.

(d) Closed-Loop Control

In some embodiments of the invention, the assays of the present invention are performed in a closed-loop. The voltage-clamp closed-loop operation comprises a control circuit producing an error signal when the membrane potential as read out by optical detection, e.g., as determined by fast voltage-sensitive dye fluorescence, ion-sensitive dye fluorescence, semiconductor nanocrystals luminescence or voltage-sensitive FRET or by extra-cellular electrodes, differs from the target membrane potential. In some embodiments, the range for the target membrane potential is about the same as the range achievable with a photoswitched ion channel, e.g., between about −100 mV and +50 mV. The error signal drives one or more of the photoswitch illumination sources so that increasing the illumination intensity of the first source produces an increase in depolarizing currents, and increasing the intensity of a second or other illumination sources produces an increase in hyperpolarizing currents.

Any algorithm, calculation, computation, or other step may be implemented according to tangible computer-readable media, non-limiting examples of which include code, logic, and instructions for performing such steps. Computer readable media may be stored in memory. Examples of tangible computer readable media include, but are not limited to, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read. One or more processors may access such memory and implement the steps therein.

In some embodiments, the closed-loop control system comprises one or more control devices. A control device may be connected to the closed-loop system and/or to other control devices as part of a network. Examples of control devices include, but are not limited to, personal computers, server computers, laptop computers; personal digital assistants (PDAs) such as a Palm-based device or Windows CE device; phones such as cellular phones or location-aware portable phones (such as GPS); a roaming device, such as a network-connected roaming device; a wireless device such as a wireless email device or other device capable of communicating wireless with a computer network; or any other type of network device that may communicate over a network and handle electronic transactions. In some embodiments, the control system may include multiple devices. In some instances, the control system may include a client-server architecture. In some embodiments, network devices may be specially programmed to perform one or more step or calculation or perform any algorithm, as described herein. The control system may further issue instructions to other components of the system, as described above, such as changing the illumination intensity of a photoswitch illumination source.

(e) Systems Comprising a Dispenser

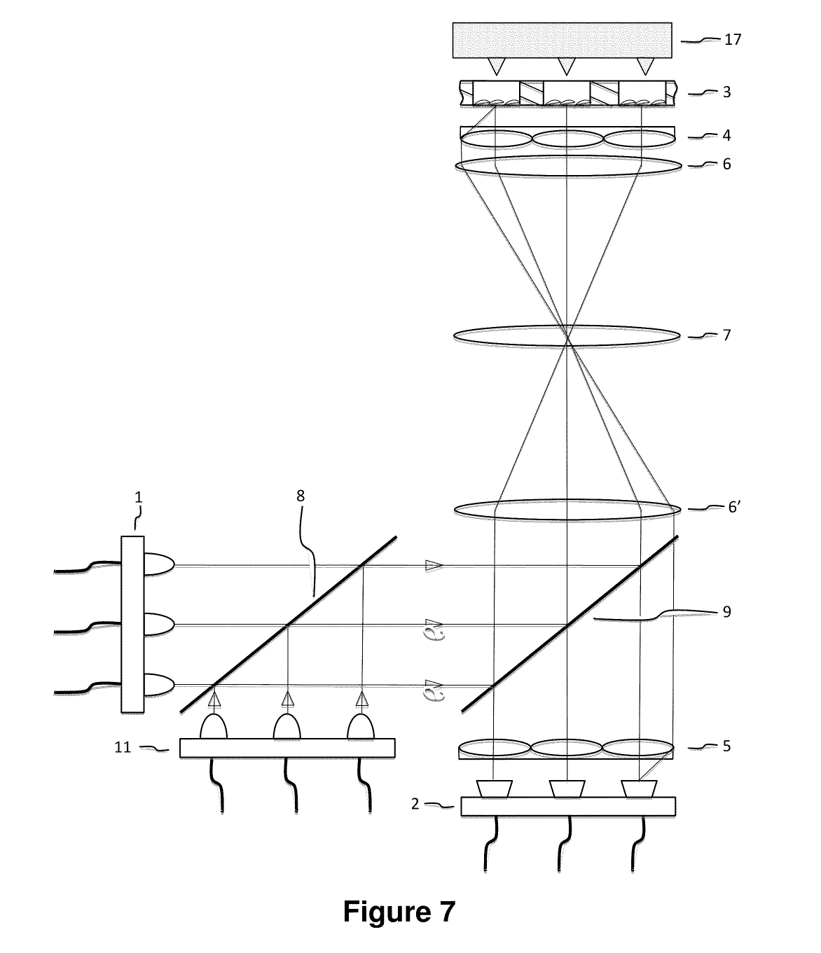

In some embodiments, assays of the invention include protocols having a reagent dispensed into the solution containing the test channel expressing cells. In some embodiments assays can be performed using Configurations 1, 1a and 2, shown in FIGS. 3, 4 and 6, respectively, wherein the system comprises a dispenser above the sample array. The dispenser can comprise one or more outlets for dispensing one or more reagents. Outlets may or may not correspond to wells or groups of wells of the sample array, such as individual wells, clusters of wells, rows of wells, or columns of wells. The dispenser can be mobile or immobile, and may be operated manually or automatically. In some embodiments, the sample array is mobile with respect to the dispenser. Mobility of the dispenser and/or sample array may be in any direction, including horizontal, vertical, and diagonal. The dispenser can be gravity driven, pressure driven, mechanically driven, or electrically driven. Examples of dispensers that may be used, and/or comprise features that may be incorporated in devices and systems of the invention, include but are not limited to the Nanodrop, Screenmaker, and Platemaker series dispensers by Innovadyne Technologies (Santa Rosa, Calif.); the Vertical Pipetting Station by Agilent Technologies (Santa Clara, Calif.); and EL, MicroFill, MicroFlo, and Precision dispensers by BioTek (Winooski, Vt.). FIG. 7 illustrates a system of the invention similar to that presented in FIG. 3, wherein a dispenser 17 is situated above the sample array.

V. Systems

In some aspects, the present invention provides systems to carry out the methods of the invention. In some embodiments, the systems are used to determine the effect of a compound on a test channel. In some embodiments, the systems comprise cells expressing a test channel and a photoswitchable ion channel, a photoswitch regulator of the photoswitchable ion channel, one or more illumination sources, and a device configurable to determine a membrane potential of the cells. The systems can be configured according to any of the embodiments and variations disclosed herein.

VI. Kits

In some aspects, the present invention provides kits for end users to carry out the methods of the invention. In some embodiments, the kits contain one or more materials useful for carrying out the methods of the invention, e.g., cells, recombinant constructs encoding test channels or photoswitchable ion channels, reagents, optical detection devices, etc. In some embodiments, the kits contain instructions for carrying out the methods of the invention. In some embodiments, the materials provided with the kits are packaged for sale.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Ion Channel Assay of a Test Compound

Cells expressing an exogenous hERG channel as the target test channel and an exogenous SPARK channel as the photoswitch-enabled ion channel are transferred to a 96 well plate. Prior to conducting the assay, cells are incubated at 37° C. with 5% CO₂ in suitable culture media. Illustrative examples of the assay are illustrated in FIG. 2. Cells are contacted with maleimid-azobenzene-quaternary ammonium, a photoswitch regulator of the SPARK channel. A test compound is added to some wells of the plate, and only carrier solution is added to other wells, the latter serving as controls. Upon exposure to light, the photoswitchable channel is activated, allowing ions to enter the cell. The test channel opens in response to the change in membrane potential resulting from the opening of the photoswitched channel. The cells are then exposed to light of another wavelength, resulting in closure of the photoswitch-enable ion channel. The change in ion flux through the channels is measured throughout the assay using the ion-sensitive dye BTC-AM, added in solution to the cells along with a solution of Tl₂SO₄. Fluorescence readings for the wells containing the test compound are then compared to those for the control wells. The effect of the test compound, if any, on the hERG channel is then inferred from the difference, if any, in the readings for flux through the channels.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining the effect of a compound on a test channel, the method comprising:
(a) providing cells expressing both the test channel and a photoswitchable ion channel;
(b) contacting the cells with a photoswitch regulator of the photoswitchable ion channel;
(c) illuminating the contacted cells with a first light source that modulates the photoswitchable ion channel; and
(d) determining the effect of the compound on the test channel.

2. The method of claim 1, wherein step (d) comprises determining a membrane potential of the cells.

3. The method of claim 1, wherein step (d) comprises determining a change in the concentration of an ion.

4. The method of claim 1, wherein the cells further express one or more proteins that extend the range of the membrane potential changes resulting from the activity of the photoswitchable channel.

5. The method of claim 1, further comprising illuminating the cells with a second light source after illuminating the cells with the first light source.

6. The method of claim 5, wherein illumination by the second light source counteracts the modulation of the photoswitchable ion channel by the first light source.

7. The method of claim 2, wherein determining the membrane potential of the cells comprises one or more of an optical measurement and an electrical measurement.

8. The method of claim 7, wherein the optical measurement comprises detecting a voltage-sensitive dye fluorescence, a voltage sensitive fluorescence resonance energy transfer (FRET), or a nanocrystal luminescence.

9. The method of claim 3, wherein determining a change in the concentration of an ion comprises detecting an ion sensitive dye fluorescence.

10. The method of claim 7, further comprising producing an error signal when the determined membrane potential differs from an expected membrane potential range.

11. A system for determining the effect of a compound on a test channel, the system comprising:
(a) cells expressing the test channel and a photoswitchable ion channel;
(b) a photoswitch regulator of the photoswitchable ion channel;
(c) one or more illumination sources; and
(d) a device configurable to determine the effect of the compound on the test channel.

12. The system of claim 11, wherein determining the effect of the compound on the test channel comprises determining a membrane potential of the cells.

13. The system of claim 11, wherein determining the effect of the compound on the test channel comprises determining a change in the concentration of an ion.

14. The system of claim 11, wherein the photoswitchable ion channel comprises a Synthetic Photoisomerizable Azobenzene-Regulated K+ (SPARK) channel or a variant thereof and the photoswitch regulator comprises Maleimide-Azobenzene-Quartenary ammonium (MAQ) or a variant thereof.

15. The system of claim 11, wherein the photoswitchable ion channel comprises a Light-activated ionotropic Glutamate Receptor (LiGluR) channel or a variant thereof and the photoswitch regulator comprises Maleimide-Azobenzene-Glutamate (MAG) or a variant thereof.

16. An optical detection device comprising:
one or more light sources;
one or more dichroic beam combiners configurable to combine light from the one or more light sources;
a sample lens array configurable to image combined light from the one or more dichroic beam combiners onto a sample array; and
a detector lens array configurable to image light from the sample array onto a detector array.

17. The device of claim 16, wherein said one or more light sources comprise one or more source arrays.

18. The device of claim 16, wherein said one or more light sources comprise two or more excitation sources, wherein each excitation source is optically coupled to separate optics

19. The device of claim 16, further comprising a dispenser configured to dispense one or more reagents to the sample array.

20. A kit comprising one or more of materials, instructions, and devices configurable and adaptable to carry out a method of any of claims 1-10.
